(19) United States
(12) Patent Application Publication
(10) Pub. No.: US 2020/0390719 A1
(43) Pub. Date: Dec. 17, 2020

(54) BIOPHOTONIC SILICONE MEMBRANES FOR TREATMENT OF SCARS

(71) Applicant: KLOX TECHNOLOGIES INC., Laval (CA)

(72) Inventors: Abdellatif CHENITE, Kirkland (CA); Stephane FAUVERGHE, Montreal (CA); Nikolaos LOUPIS, Athens (GR); Remigio PIERGALLINI, San Benedetto del Tronto (IT)

(73) Assignee: Klox Technologies Inc., Laval, QC (CA)

(21) Appl. No.: 16/643,101

(22) PCT Filed: Aug. 28, 2018
(86) PCT No.: PCT/CA2018/051035

§ 371 Date: Feb. 28, 2020

Related U.S. Application Data
(60) Provisional application No. 62/550,982, filed on Aug. 28, 2017.

(51) Int. Cl. A61K 9/70; A61L 15/48; A61L 15/26; C08L 83/04; A61L 15/58; C09B 11/28; A61N 5/06

(52) U.S. Cl. CPC A61K 9/70; A61L 15/48; A61L 15/26; C08L 83/04; A61L 15/58; A61N 2005/0662; A61N 5/062; A61N 5/0616; A61N 2005/0645; A61L 2300/442; C09B 11/28

(57) ABSTRACT

The present technology generally provides biophotonic silicone membranes and methods useful in the management of scars. In particular, the biophotonic silicone membranes of the present technology are useful in preventing and/or treating post-surgical scar formation.

### CROSS-REFERENCE TO RELATED APPLICATION

This application claims the benefit of and priority to U.S. provisional patent application No. 62/550,982, filed on Aug. 28, 2017, the content of which is herein incorporated in entirety by reference.

### TECHNICAL FIELD

The present technology generally relates to biophotonic silicone membranes and to their use in methods of management and/or treatment of scars.

### BACKGROUND INFORMATION

Skin is the largest organ of the human body with an average surface of 1.8 square meters. Of its many amazing properties is its ability to heal in response to different external aggressions. However, the healing process will often lead to the formation of keloids and/or hypertrophic scars, abnormal responses to injury. Many invasive and non-invasive options are available to the clinician. Invasive treatment options include intralesional injections of corticosteroids and/or 5-fluorouracil, cryotherapy, radiotherapy, laser therapy and surgical excision. Non-invasive treatment options include use of creams, ointments and/or gels comprising agents that enhance treatment of scars such as for example, vitamin E.

Other general scar preventative measures include avoiding sun exposure, compression therapy, taping and the use of moisturizers. All of these options may be used alone or as part of combination therapy. However, reduction of scarring represents a significant and largely unmet medical need in a wide variety of clinical settings.

Biophotonic compositions and their use in treatment of scars have been proposed in International Application Publication No. WO 2015/189712, incorporated herein by reference.

However, the scar treatment products and methods known to date may not be efficient at treating all scar types. As such, there remains a need in the art for additional scar treatment products and methods that will allow patients and health care practitioners to decide on the most efficient scar product/method treatment for a given type of scar. It is thus an object of the present disclosure to provide new and improved biophotonic silicone membranes useful in methods for treatment of scars.

### SUMMARY OF THE DISCLOSURE

The present disclosure provides biophotonic silicone membranes useful in phototherapy for treating scars.

In one embodiment, the biophotonic silicone membrane of the present technology comprises a silicone phase, for example comprising a soft silicone and a surfactant phase, wherein the surfactant phase comprises at least one light-absorbing molecule solubilized in a surfactant.

In one embodiment, the biophotonic silicone membrane of the present technology comprises a silicone phase, for example comprising a soft adhesive silicone and a surfactant phase, wherein the surfactant phase comprises at least one light-absorbing molecule solubilized in a surfactant.

In some implementations, the surfactant phase does not include triethanolamine (TEA).

In some embodiments the biophotonic silicone membrane comprises an outer coating layer including soft adhesive silicone. In some embodiments, the silicone-based biophotonic membrane of the present technology emits fluorescence at a wavelength and intensity that diminishes or prevents scarring.

In some embodiments, the biophotonic silicone membrane of the present disclosure comprises an adherent (e.g., adhesive) side and a non-adherent (e.g., non-adhesive) side.

In some embodiments, the present technology also relates to a method for management of a scar, such as, e.g., post-surgical scars, in a subject in need thereof, the method comprising: a) placing the biophotonic silicone membrane of the present technology on or over a target skin tissue, and b) illuminating the biophotonic silicone membrane with light having a wavelength that overlaps with an absorption spectrum of the at least one light-absorbing molecule.

In some embodiments, the present technology also relates to a method for preventing and/or treating a scar, such as, e.g., post-surgical scars, in a subject in need thereof, the method comprising: a) placing the biophotonic silicone membrane of the present technology on or over a target skin tissue, and b) illuminating the biophotonic silicone membrane with light having a wavelength that overlaps with an absorption spectrum of the at least one light-absorbing molecule.

In some embodiments, steps a) and b) are performed at least once weekly (i.e., one time per week). In some embodiments, steps a) and b) are performed at least twice weekly (i.e., two times per week). In some embodiments, the light in step b) is illuminated for 5 minutes at two consecutive intervals. In some embodiments, the two consecutive intervals are separated by a period of 1 to 2 minutes without illumination.

In one embodiment, the method is useful for preventing scar formation on a target skin tissue of a subject, wherein the target skin tissue is a post-surgical skin tissue (e.g., breast tissue after a bilateral breast reduction). In one embodiment, the method is useful for treating a scar (e.g., reducing or diminishing scar formation, or reducing severity of a scar). In one embodiment, the subject has undergone a bilateral breast reduction procedure.

In one embodiment, the scar to be treated or prevented from formation is any one or more of a hypertrophic scar, a keloid, a linear scar, a sunken scar, or a stretched scar on a subject. In some embodiments, the subject is a human subject or a veterinary subject.

In certain embodiments of the method, the biophotonic silicone membrane is left in place after illumination. In certain embodiments, the biophotonic silicone membrane is re-illuminated. In one embodiment, the biophotonic silicone membrane is left in place after illumination. In some embodiments, the light-absorbing molecule at least partially photobleaches during or after illumination. In some embodiments, the light-absorbing molecule photobleaches after illumination. In certain embodiments, the biophotonic silicone membrane is illuminated until the light-absorbing molecule is at least partially photobleached.

In certain embodiments of any of the foregoing or following, the light has a peak wavelength between about 400 nm and about 750 nm. The light may have a peak wavelength between about 400 nm and about 500 nm. In certain embodiments of any of the foregoing or following, the light is from a direct light source such as a lamp. The lamp may be an LED lamp. In certain embodiments, the light is from an ambient light source. In some embodiments, the light-absorbing molecule can absorb and/or emit light in the visible range.

In certain embodiments of any of the foregoing or following, the biophotonic silicone membrane is illuminated by a direct light source for about 1 minute to greater than 75 minutes, about 1 minute to about 75 minutes, about 1 minute to about 60 minutes, about 1 minute to about 55 minutes, about 1 minute to about 50 minutes, about 1 minute to about 45 minutes, about 1 minute to about 40 minutes, about 1 minute to about 35 minutes, about 1 minute to about 30 minutes, about 1 minute to about 25 minutes, about 1 minute to about 20 minutes, about 1 minute to about 15 minutes, about 1 minute to about 10 minutes, or about 1 minute to about 5 minutes.

In some embodiments, the surfactant phase of the biophotonic silicone membrane is emulsified in the silicone phase. In certain embodiments, the silicone phase is a continuous phase. In some embodiments, the surfactant is a block copolymer. The block copolymer may comprise at least one hydrophobic block and at least one hydrophilic block. In some embodiments the surfactant is thermogellable.

In certain embodiments of any of the foregoing or following, the surfactant comprises at least one sequence of polyethylene glycol-polypropylene glycol ((PEG)-(PPG)). In a further embodiment the surfactant is a triblock copolymer or poloxomer of the formula (PEG)-(PPG)-(PEG). In yet another embodiment, the surfactant is Pluronic F127.

In certain embodiments of any of the foregoing or following, the surfactant comprises at least one sequence of polyethylene glycol-polylactic acid ((PEG)-(PLA)). In some embodiments the surfactant comprises at least one sequence of polyethyelene glycol-poly(lactic-c-glycolic acid) ((PEG)-(PLGA)). In some embodiments the surfactant comprises at least one sequence of polyethyelene glycol-polycaprolactone ((PEG)-(PCL)). In a further embodiment the surfactant is a triblock copolymer or poloxomer of the formula A-B-A or B-A-B, wherein A is PEG and B is PLA or PLGA or PCL.

In certain embodiments of any of the foregoing or following, the silicone phase comprises silicone.

In certain embodiments, the silicone may be a silicone elastomer. In certain embodiments, the silicone comprises a polydimethylsiloxane. In certain embodiments, the silicone comprises MED-6360. In certain embodiments the silicone comprises a mixture of MED-6360 and MED-4011 or MED-6015. In a further embodiment the silicone comprises a mixture of about 30% MED-6360 and about 70% MED-4011. In certain embodiments, the mixture of MED-6360 and MED-4011 provides for a biophotonic membrane composition in a membrane form having an elasticity and adhesiveness which may be well suited to skin applications. Specifically, the elasticity may allow for a greater ease of manipulation of the silicone-based biophotonic membrane, and the adhesiveness may allow for the membrane to stay where it is placed during a treatment procedure as may be provided for in the present disclosure.

In certain embodiments of any of the foregoing or following, the silicone phase comprises silicone. In certain embodiments, the silicone is a silicone elastomer comprising: an organopolysiloxane having silicon-bonded alkenyl groups (e.g., dimethylsiloxane capped at both molecular termini with vinyldimethylsilyl groups); (B) an organohydrogensiloxane having an average of two or more silicon-bonded hydrogen atoms in the molecule (e.g., dimethylsiloxane and methyl hydrogen siloxane capped at both molecular termini with trimethylsilyl groups); (C) an inorganic filler (e.g., Fumed silica); and (D) a filler treatment agent which includes an alkenyl-containing group (e.g., hexamethyldisilazane). In other embodiments, the filler treating agent can be a mixture of (D1) an alkenyl-free organosilane, organosilazane, organosilanol, alkoxyorganosilane, or any combination thereof and (D2) an alkenyl-containing organosilane, organosilazane, organosilanol, alkoxyorganosilane, or any combination thereof, e.g., the filler treating agent can be a mixture of (D1) alkenyl-free organosilane or organosilazane and (D2) alkenyl-containing organosilane or organosilazane. In certain embodiments, the silicone is a silicone elastomer having: (i) a Shore-A hardness of from about 20 to about 45 as measured in accordance with ASTM D2240 using a type A durometer hardness tester; (ii) a breaking elongation of at least about 800% as measured in accordance with ASTM D412; and (iii) a tensile strength of at least about 15.0 MPa.

In certain embodiments, the silicone phase is formed from a composition comprising: (A) 100 parts of an organopolysiloxane having alkenyl radicals; (B) 0.3 to 20 parts of an organohydrogensiloxane having an average of two or more silicon-bonded hydrogen atoms in the molecule; (C) 10 to 50 parts of an inorganic filler; and (D) 0.05 to 20 parts of a filler treatment agent which includes an alkenyl-containing group.

In certain embodiments, the biophotonic silicone membrane of the present technology comprises an outer coating including soft adhesive silicone (such as but not limited to: MED-6360) that confers enhanced adhesiveness. In some embodiments, the soft adhesive silicone is coated on one side of the biophotonic silicone membrane.

In certain embodiments of any of the foregoing or following, biophotonic silicone membrane comprises 80 wt % silicone phase and about 20 wt % surfactant phase. In some embodiments the biophotonic silicone membrane comprises a silicone phase/surfactant phase wt % composition of about 60/40 wt %, or about 65/55 wt %, or about 70/30 wt %, or about 75/25 wt %, or about 80/20 wt %, or about 85/15 wt % or about 90/10 wt %.

In certain embodiments of any of the foregoing or following, the at least one light-absorbing molecule is water soluble and is solubilized in the surfactant phase. The at least one light-absorbing molecule may be a fluorophore. In certain embodiments, the light-absorbing molecule can absorb and/or emit light. In some embodiments, the light absorbed and/or emitted by the light-absorbing molecule is in the visible range of the electromagnetic spectrum. In some embodiments, the light absorbed and/or emitted by the light-absorbing molecule is in the range of about 400 nm to about 750 nm. In certain embodiments, the light-absorbing molecule can emit light from around 500 nm to about 700 nm. In some embodiments, the light-absorbing molecule or the fluorophore is a xanthene dye. The xanthene dye may be selected from Eosin Y, Eosin B, Erythrosine B, Fluorescein, Rose Bengal and Phloxin B.

In certain embodiments of any of the foregoing or following, the surfactant phase of the biophotonic silicone membrane further comprises a stabilizer. In further embodiments the stabilizer comprises gelatin, hydroxyethyl cellulose ether (HEC), carboxymethyl cellulose (CMC) or any other thickening agent.

In certain embodiments of any of the foregoing or following, the biophotonic silicone membrane is at least substantially translucent. The biophotonic silicone membrane may be transparent. In some embodiments, the biophotonic silicone membrane has a translucency of at least about 40%, about 50%, about 60%, about 70%, or about 80% in a visible range. Preferably, the light transmission through the biophotonic silicone membrane is measured in the absence of the at least one light-absorbing molecule.

In certain embodiments of any of the foregoing or following, the biophotonic silicone membrane has a thickness of about 0.1 mm to about 50 mm, about 0.5 mm to about 20 mm, or about 1 mm to about 10 mm, or about 1 mm to about 5 mm.

In certain embodiments of any of the foregoing or following, the biophotonic silicone membrane has a removeable cover for covering one or both sides of the membrane. The removeable cover may be peelable. The removeable cover may comprise a sheet or a film of material, such as paper or foil. In certain embodiments, the removeable cover is opaque and can protect the membrane from illumination until the treatment time. The cover may be partially removeable. In certain embodiments, the cover may be re-applicable to the membrane surface, such as after a treatment time, in order to protect the membrane from further illumination in between treatments.

In certain embodiments of any of the foregoing or following, the surfactant phase is homogenously distributed within the silicone phase and is nano and/or micro-sized. It can be considered as micro-emulsified. The surfactant phase is not visibly detectable by eye. In other words, the membrane appears by eye as one phase.

In certain embodiments, the biophotonic silicone membrane comprises pores (e.g., holes). In some embodiments, the membrane is non-adherent on both sides, allowing the membrane to be placed on the target site of a subject on either side. In some embodiments, the membrane is non-adherent on one-side, and adherent on the opposite side. In further embodiments, the method further comprises placing an absorbent dressing over the pores of the biophotonic silicone membrane allowing, e.g., the dressing to absorb material that passes from the treatment site (wound) through the pores.

In some embodiments, the biophotonic silicone membrane comprises an outer coating consisting of a silicone elastomer, such as, but not limited to: MED-6360 (soft adhesive/adherent silicone), that confers enhanced adhesiveness. In some embodiments, the outer coating has a thickness in a range of about 50μμm to about 500 μm.

The present disclosure also provides a kit comprising a biophotonic silicone membrane having a silicone phase and a surfactant phase, and wherein the surfactant phase comprises at least one light-absorbing molecule solubilized in a surfactant; and instructions for performing any of the methods described herein. In some embodiments, the kit comprises a multi-LED lamp.

### BRIEF DESCRIPTION OF THE DRAWINGS

Further aspects and advantages of the present technology will become better understood with reference to the description in association with the following in which:

FIG. 1 illustrates an overview of the clinical study design;

FIGS. 2A-2E are 3D-photographs of the two treating areas of wounds treated with a biophotonic silicone membrane (BSM) according to one embodiment of the present technology and with Standard of Care consisting of massaging the wound with Vitamin E cream (Vitamin E);

FIGS. 3A-3H are graphs showing the results of a treatment using a biophotonic silicone membrane (BSM) according to one embodiment of the present technology as assessed on a Vancouver Scar Scale (VSS) compared to a treatment with Standard of Care consisting of massaging the wound with Vitamin E cream (Vit E); FIG. 3A: Pain; FIG. 3B: Itchiness; FIG. 3C: Color; FIG. 3D: Stiffness; FIG. 3E: Thickness; FIG. 3F: Irregularity; FIG. 3G: Total score; and FIG. 3H: Overall opinion;

FIGS. 4A-4H are graphs showing the results of a treatment using a biophotonic silicone membrane (BSM) according to one embodiment of the present technology as assessed on a Patient and Observer Scar Assessment Scale (POSAS) compared to a treatment with Standard of Care consisting of massaging the wound with Vitamin E cream (Vit E) ; FIG. 4A: Vascularity; FIG. 4B: Pigmentation; FIG. 4C: Thickness; FIG. 4D: Relief; FIG. 4E: Pliability; FIG. 4F: Surface area; FIG. 4G: Total score; and FIG. 4H: Overall opinion;

FIG. 5 are pictures showing modulation of scar morphology and wound closure by a biophotonic silicone membrane according to one embodiment of the present technology. The wounds were treated as indicated twice a week during the first 6 weeks or left untreated (control). Wounds were monitored by digital photography weekly after grafting.

FIGS. 6A-6C are graphs showing the effect of treatment with a biophotonic silicone membrane according to one embodiment of the present technology on reepithelization and reduced scar thickness and vascularity. Epidermis (6A) and dermis (6B) thickness, blood vessel numbers (6C) were determined. Bar graphs represent the mean±SEM of 5 or 6 mice/group. (*p≤0.05; **p≤0.01).

FIG. 7 is a graph showing the effect of a treatment with a biophotonic silicone membrane according to one embodiment of the present technology on collagen deposition. Collagen deposition of xenografts harvested from mice treated as indicated at 1, 2, 3 months (m) after treatment was quantified by 4-hydroxyproline assessment. Bar graphs represent the mean±SEM of 5 or 6 mice/group, each performed in triplicate. The data is displayed by ng of 4-hydroxyproline per mg of dry tissue referring to a standard curve. (*p≤005; **p≤0.01).

FIG. 8 is a graph showing the effect of a treatment with a biophotonic silicone membrane according to one embodiment of the present technology on myofibroblast accumulation. (A) αSMA immunostaining of xenografts harvested from mice treated as indicated at 1, 2, 3 months (m) post-treatment to evaluate myofibroblast formation over time during scarring. Endothelial cells around blood vessels and myofibroblasts were all stained by anti-αSMA antibody, but it is very easy to distinguish the myofibroblasts (arrows) from endothelial cells (stars). Scale bar, 50 μm. (B) Myofibroblasts were counted in five high power fields (HPFs). Bar graphs represent the mean±SEM of 5 or 6 mice/group. (*, p≤0.05; **p≤0.01).

FIG. 9 is a graph showing the effect of a treatment with a biophotonic silicone membrane according to one embodiment of the present technology on mast cells. (A) Mast cells in the xenografts harvested from mice treated as indicated at 1, 2, 3 months (m) after treatment were stained by Toluidine blue to evaluate mast cell recruitment (arrows) over time during scar formation. Scale bar is 50 μm. (B) Graphs represent the mean±SEM of 5 or 6 mice/group. *Control vs Light; #Control vs Membrane; $ Control vs Gel. (*, #, $ p≤0.05).

FIG. 10 is a graph showing the effect of a treatment with a biophotonic silicone membrane according to one embodiment of the present technology on fibrotic factor production. Immunostaining of connective tissue growth factor (CTGF) from xenografts harvested from mice treated as indicated at 1, 2, 3 months (m) post-treatment. Mouse number is 5 or 6 in each group. Scale bar is 100 μm.

### DETAILED DESCRIPTION

Three distinct phases involved in the pathophysiology of excessive scar formation have been described: inflammation, proliferation and remodelling. In normal scar healing, during the inflammation phase, platelet degranulation will be responsible for the release and activation of an array of different potent cytokines which will serve as chemotactic agents to recruit macrophages, neutrophils, epithelial cells and fibroblasts. In normal conditions, a balance will be achieved between new tissue biosynthesis and degradation mediated by apoptosis and remodeling of the extracellular matrix. In excessive scarring, a persistent inflammation, caused by an increased secretion of different factors (e.g., TGF-β1, TGF-β2, PDGF, IGF-1, IL-4 and IL-10) might lead to an excessive collagen synthesis or deficient matrix degradation and remodeling. Scars are classified into different categories, based on the nature of the injury having caused the scar, its clinical characteristics and its appearance. Flat or pale scars (known as linear scars) are the most common type of scar and result from the body's natural healing process. Initially, these scars may be red or dark and raised after the wound has healed but they will eventually become paler and flatten naturally over time, resulting in a flat, pale scar. This process can take up to two years and there will always be some visible evidence of the original wound. Hypertrophic scars are more common in young people and people with darker skin. When a normal wound heals, the body produces new collagen fibres at a rate which balances the breakdown of old collagen. Hypertrophic scars are red and thick and may be itchy or painful. They do not extend beyond the boundary of the original wound but may continue to thicken for up to 6 months. They usually improve over the next one to two years but may cause distress due to their appearance or the intensity of the itching, also restricting movement if they are located close to a joint. It is not possible to completely prevent hypertrophic scars. Similar to hypertrophic scars, keloids are the result of an imbalanced collagen production in a healing wound. Unlike hypertrophic scars, keloids grow beyond the boundary of the original wound and can continue to grow indefinitely. They may be itchy or painful and most will not improve in appearance over time. Keloid scars can result from any type of injury to the skin, including scratches, injections, insect bites and tattoos. Some parts of the body are more sensitive to the development of keloids, such as ears, chest, shoulders and back. As with hypertrophic scarring, people who have developed one keloid scar are more prone to this condition in the future. Sunken scars are recessed into the skin. They may be due to the skin being attached to deeper structures (such as muscles) or to loss of underlying fat. They are usually the result of an injury. A very common cause of sunken scarring is acne or chicken pox which can result in a pitted appearance, although acne scarring is not always sunken in appearance and can even become keloid. Finally, stretched scars occur when the skin around a healing wound is put under tension during the healing process. This type of scarring may follow injury or surgery. Initially, the scar may appear normal but can widen and thin over a period of weeks or months. This can occur where the skin is close to a joint and is stretched during movement or may be due to poor healing due to general ill health or malnutrition.

Different tools are available to evaluate scars. The Patient and Observer Scar Assessment Scale (POSAS) is designed to be used by both the clinician and the patient. The clinician will assess the scar looking at vascularity, pigmentation, thickness, relief, pliability and importance of surface area whereas the patient will look after pain, itching, color, stiffness, thickness, contour irregularities and overall opinion. The Vancouver Scar Scale (VSS) is another validated scale used for scars assessment. Methods for documenting scar development and response to treatment are available, including various photography techniques as well as computerized digital camera medical devices, useful to make comparisons and follow-ups over time.

In one embodiment, the present disclosure provides biophotonic silicone membrane for preventing and/or treating scars as well of methods of using such biophotonic silicone membrane in the prevention and/or treatment of scars, for example post-surgical scars. The membranes and methods of the present disclosure combine the beneficial effects of topical silicone compositions with the photobiostimulation induced by the fluorescent light generated by the light-absorbing molecule(s) upon illumination of the biophotonic silicone membranes. The expressions “biophotonic silicone composition”, “biophotonic silicone membrane”, and “biophotonic membrane composition” are used interchangeably.

Before continuing to describe the present disclosure in further detail, it is to be understood that this disclosure is not limited to specific compositions or process steps, as such may vary. It must be noted that, as used in this specification and the appended claims, the singular form “a”, “an” and “the” include plural referents unless the context clearly dictates otherwise.

As used herein, the term “about” in the context of a given value or range refers to a value or range that is within 20%, preferably within 10%, and more preferably within 5% of the given value or range.

It is convenient to point out here that “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

“Biophotonic” means the generation, manipulation, detection and application of photons in a biologically relevant context. In other words, biophotonic compositions exert their physiological effects primarily due to the generation and manipulation of photons.

“Topical application” or “topical uses” means application to body surfaces, such as the skin, mucous membranes, vagina, oral cavity, internal surgical wound sites, and the like.

“Emulsion” shall be understood as referring to a temporary or permanent dispersion of one liquid phase within a second liquid phase. Generally one of the phases is an aqueous solution, and the other a water-immiscible liquid. The water-immiscible liquid is generally referred to as the continuous phase. In this disclosure, the continuous phase comprises a silicone and is referred to as a silicone phase. Moreover, in this disclosure, the aqueous phase comprises a surfactant and is referred to as a surfactant phase.

Expressions “light-absorbing molecule”, “light-activated molecule”, “chromophore” and “photoactivator” are used herein interchangeably. A light-absorbing molecule means a chemical compound, when contacted by light irradiation, is capable of absorbing the light. The light-absorbing molecule readily undergoes photoexcitation and can transfer its energy to other molecules or emit it as light (fluorescence).

“Photobleaching” or “photobleaches” means the photochemical destruction of a light-absorbing molecule. A light-absorbing molecule may fully or partially photobleach.

The term “actinic light” is intended to mean light energy emitted from a specific light source (e.g., lamp, LED, or laser) and capable of being absorbed by matter (e.g. the light-absorbing molecule or photoactivator). Terms “actinic light” and “light” are used herein interchangeably. In a preferred embodiment, the actinic light is visible light.

The term “preventing” or “prevention” as used herein in the context of preventing a scar or prevention of a scar, refers to eliminating, ameliorating, decreasing or reducing a scar or development of a scar. The term “treating” or “treatment” as used herein the context of treating a scar or treatment of a scar, refers to having a therapeutic effect and at least partially alleviating or abrogating or ameliorating a scar.

Features and advantages of the subject matter hereof will become more apparent in light of the following detailed description of selected embodiments. As will be realized, the subject matter disclosed and claimed is capable of modifications in various respects, all without departing from the scope of the claims. Accordingly, the drawings and the description are to be regarded as illustrative in nature and not as restrictive and the full scope of the subject matter is set forth in the claims.

Biophotonic Silicone Membranes

The present disclosure provides, in a broad sense, biophotonic silicone membranes and methods of using the biophotonic silicone membranes. Biophotonic silicone membranes can be, in a broad sense, activated by light (e.g., photons) of specific wavelength. A biophotonic silicone membrane according to various embodiments of the present disclosure comprises a silicone phase and a surfactant phase, with at least one light-absorbing molecule solubilized in the surfactant phase. In some embodiments, the surfactant phase is emulsified in the silicone phase. In some embodiments, the surfactant phase is emulsified in the silicone phase, and a further coating of silicone layer is provided to confer enhanced adhesiveness.

The light-absorbing molecule in the biophotonic silicone membrane may be activated by light. This activation accelerates the dispersion of light energy, leading to light carrying on a therapeutic effect on its own, and/or to the photochemical activation of other agents contained in the membrane. This may lead to the breakdown of the light-absorbing molecule and, in some embodiments, ensure that the biophotonic silicone membrane is for single-use.

When a light-absorbing molecule absorbs a photon of a certain wavelength, it becomes excited. This is an unstable condition and the molecule tries to return to the ground state, giving away the excess energy. For some light-absorbing molecules, it is favorable to emit the excess energy as light when returning to the ground state. This process is called fluorescence. The peak wavelength of the emitted fluorescence is shifted towards longer wavelengths compared to the absorption wavelengths due to loss of energy in the conversion process. This is called the Stokes' shift. In the proper environment (e.g., in a biophotonic composition) much of this energy is transferred to the other components of the biophotonic composition or to the treatment site directly. Without being bound to theory, it is thought that fluorescent light emitted by photoactivated light-absorbing molecules may have therapeutic properties due to its femto-, pico-, or nano-second emission properties which may be recognized by biological cells and tissues, leading to favourable biomodulation. Furthermore, generally, the emitted fluorescent light has a longer wavelength and hence a deeper penetration into the tissue than the activating light. Irradiating tissue with such a broad range of wavelength, including in some embodiments the activating light which passes through the composition, may have different and complementary effects on the cells and tissues. In other words, light-absorbing molecules are used in the biophotonic silicone membranes of the present disclosure for therapeutic effect on tissues. This is a distinct application of these photoactive agents and differs from the use of light-absorbing molecules as simple stains or as catalysts for photo-polymerization.

The biophotonic silicone membranes of the present disclosure are used topically as a dressing or a membrane adhesive onto an affected area of the skin. In some embodiments, the biophotonic silicone membranes are cohesive. The cohesive nature of these biophotonic silicone membranes may provide ease of removal from the site of treatment and hence provide for a convenient ease of use. Additionally or alternatively, the biophotonic silicone membranes of the present disclosure have functional (e.g., sticky or adhesive) and structural properties and these properties may also be used to define and describe the membranes. Individual components of the biophotonic silicone membrane of the present disclosure, including light-absorbing molecules, surfactants, silicone, and other optional ingredients, are detailed below.

Light-Absorbing Molecules

Suitable light-absorbing molecules can be fluorescent compounds (or stains) (also known as “fluorochromes” or “fluorophores”). Other dye groups or dyes (biological and histological dyes, food colorings, carotenoids, and other dyes) can also be used. Suitable photoactivators can be those that are Generally Regarded As Safe (GRAS). Advantageously, photoactivators which are not well tolerated by the skin or other tissues can be included in the biophotonic composition of the present disclosure, as in certain embodiments, the photoactivators are encapsulated within the surfactant phase of the emulsion in the silicone continuous phase. In certain embodiments, the light-absorbing molecule is one which undergoes partial or complete photobleaching upon application of light. In some embodiments, the light-absorbing molecule absorbs at a wavelength in the range of the visible spectrum, such as at a wavelength of about 380-800 nm, 380-700 nm, 400-800 nm, or 380-600 nm. In other embodiments, the light-absorbing molecule absorbs at a wavelength of about 200-800 nm, 200-700 nm, 200-600 nm or 200-500 nm. In one embodiment, the light-absorbing molecule absorbs at a wavelength of about 200-600 nm. In some embodiments, the light-absorbing molecule absorbs light at a wavelength of about 200-300 nm, 250-350 nm, 300-400 nm, 350-450 nm, 400-500 nm, 450-650 nm, 600-700 nm, 650-750 nm or 700-800 nm. It will be appreciated to those skilled in the art that optical properties of a particular light-absorbing molecule may vary depending on the light-absorbing molecule's surrounding medium. Therefore, as used herein, a particular light-absorbing molecule's absorption and/or emission wavelength (or spectrum) corresponds to the wavelengths (or spectrum) measured in a biophotonic silicone membrane of the present disclosure.

The biophotonic silicone membrane disclosed herein may include at least one additional light-absorbing molecule or second light-absorbing molecule. Combining light-absorbing molecules may increase photo-absorption by the combined dye molecules and enhance absorption and photo-biomodulation selectivity. This creates multiple possibilities of generating new photosensitive, and/or selective light-absorbing molecules mixtures. Thus, in certain embodiments, biophotonic silicone membranes of the disclosure include more than one light-absorbing molecule, and when illuminated with light, energy transfer can occur between the light-absorbing molecules. This process, known as resonance energy transfer, is a widely prevalent photophysical process through which an excited ‘donor’ light-absorbing molecule (also referred to herein as first light-absorbing molecule) transfers its excitation energy to an ‘acceptor’ light-absorbing molecule (also referred to herein as second light-absorbing molecule). The efficiency and directedness of resonance energy transfer depends on the spectral features of donor and acceptor light-absorbing molecules. In particular, the flow of energy between light-absorbing molecules is dependent on a spectral overlap reflecting the relative positioning and shapes of the absorption and emission spectra. More specifically, for energy transfer to occur, the emission spectrum of the donor light-absorbing molecule must overlap with the absorption spectrum of the acceptor light-absorbing molecule. Energy transfer manifests itself through decrease or quenching of the donor emission and a reduction of excited state lifetime accompanied also by an increase in acceptor emission intensity. To enhance the energy transfer efficiency, the donor light-absorbing molecule should have good abilities to absorb photons and emit photons. Furthermore, the more overlap there is between the donor light-absorbing molecule's emission spectra and the acceptor light-absorbing molecule's absorption spectra, the better a donor light-absorbing molecule can transfer energy to the acceptor light-absorbing molecule. Accordingly, in embodiments comprising a mixture of light-absorbing molecules, the first light-absorbing molecule has an emission spectrum that overlaps at least about 80%, 50%, 40%, 30%, 20% or 10% with an absorption spectrum of the second light-absorbing molecule. In one embodiment, the first light-absorbing molecule has an emission spectrum that overlaps at least about 20% with an absorption spectrum of the second light-absorbing molecule. In some embodiments, the first light-absorbing molecule has an emission spectrum that overlaps at least 1-10%, 5-15%, 10-20%, 15-25%, 20-30%, 25-35%, 30-40%, 35-45%, 50-60%, 55-65%, 60-70% or 70-80% with an absorption spectrum of the second light-absorbing molecule. Percent (%) spectral overlap, as used herein, means the % overlap of a donor light-absorbing molecule's emission wavelength range with an acceptor light-absorbing molecule's absorption wavelength rage, measured at spectral full width quarter maximum (FWQM). In some embodiments, the second light-absorbing molecule absorbs at a wavelength in the range of the visible spectrum. In certain embodiments, the second light-absorbing molecule has an absorption wavelength that is relatively longer than that of the first light-absorbing molecule within the range of about 50-250 nm, 25-150 nm or 10-100 nm.

The light-absorbing molecule may be present in an amount of about 0.001-40% per weight of the membrane or of the surfactant phase. In certain embodiments, the at least one light-absorbing molecule is present in an amount of about 0.001-3%, 0.001-0.01%, 0.005-0.1%, 0.1-0.5%, 0.5-2%, 1-5%, 2.5-7.5%, 5-10%, 7.5-12.5%, 10-15%, 12.5-17.5%, 15-20%, 17.5-22.5%, 20-25%, 22.5-27.5%, 25-30%, 27.5-32.5%, 30-35%, 32.5-37.5%, or 35-40% per weight of the biophotonic silicone membrane.

In certain embodiments, the at least one light-absorbing molecule is present in an amount of about 0.001-3%, 0.001-0.01%, 0.005-0.1%, 0.1-0.5%, 0.5-2%, 1-5%, 2.5-7.5%, 5-10%, 7.5-12.5%, 10-15%, 12.5-17.5%, 15-20%, 17.5-22.5%, 20-25%, 22.5-27.5%, 25-30%, 27.5-32.5%, 30-35%, 32.5-37.5%, or 35-40% of the surfactant phase.

When present, the second light-absorbing molecule may be present in an amount of about 0.001-40% per weight of the biophotonic silicone membrane or of the surfactant phase. In certain embodiments, the second light-absorbing molecule is present in an amount of about 0.001-3%, 0.001-0.01%, 0.005-0.1%, 0.1-0.5%, 0.5-2%, 1-5%, 2.5-7.5%, 5-10%, 7.5-12.5%, 10-15%, 12.5-17.5%, 15-20%, 17.5-22.5%, 20-25%, 22.5-27.5%, 25-30%, 27.5-32.5%, 30-35%, 32.5-37.5%, or 35-40% per weight of the biophotonic silicone membrane or of the surfactant phase. In certain embodiments, the total weight per weight of light-absorbing molecule or combination of light-absorbing molecules may be in the amount of about 0.005-1%, 0.05-2%, 1-5%, 2.5-7.5%, 5-10%, 7.5-12.5%, 10-15%, 12.5-17.5%, 15-20%, 17.5-22.5%, 20-25%, 22.5-27.5%, 25-30%, 27.5-32.5%, 30-35%, 32.5-37.5%, or 35-40% per weight of the biophotonic silicone membrane or of the surfactant phase.

The concentration of the light-absorbing molecule to be used can be selected based on the desired intensity and duration of the biophotonic activity from the biophotonic silicone membrane, and on the desired medical or cosmetic effect. For example, some dyes such as xanthene dyes reach a ‘saturation concentration’ after which further increases in concentration do not provide substantially higher emitted fluorescence. Further increasing the light-absorbing molecule concentration above the saturation concentration can reduce the amount of activating light passing through the matrix. Therefore, if more fluorescence is required for a certain application than activating light, a high concentration of light-absorbing molecule can be used. However, if a balance is required between the emitted fluorescence and the activating light, a concentration close to or lower than the saturation concentration can be chosen. Suitable light-absorbing molecules that may be used in the biophotonic silicone compositions of the present disclosure include, but are not limited to the following:

Chlorophyll dyes—Exemplary chlorophyll dyes include but are not limited to chlorophyll a; chlorophyll b; chlorophyllin; bacteriochlorophyll a; bacteriochlorophyll b; bacteriochlorophyll c; bacteriochlorophyll d; protochlorophyll; protochlorophyll a; amphiphilic chlorophyll derivative 1; and amphiphilic chlorophyll derivative 2.

Xanthene derivatives—Exemplary xanthene dyes include, but are not limited to, eosin B, eosin B (4′,5′-dibromo,2′,7′-dinitr-o-fluorescein, dianion); Eosin Y; eosin Y (2′,4′,5′,7′-tetrabromo-fluoresc-ein, dianion); eosin (2′,4′,5′,7′-tetrabromo-fluorescein, dianion); eosin (2′,4′,5′,7′-tetrabromo-fluorescein, dianion) methyl ester; eosin (2′,4′,5′,7′-tetrabromo-fluorescein, monoanion) p-isopropylbenzyl ester; eosin derivative (2′,7′-dibromo-fluorescein, dianion); eosin derivative (4′,5′-dibromo-fluorescein, dianion); eosin derivative (2′,7′-dichloro-fluorescein, dianion); eosin derivative (4′,5′-dichloro-fluorescein, dianion); eosin derivative (2′,7′-diiodo-fluorescein, dianion); eosin derivative (4′,5′-diiodo-fluorescein, dianion); eosin derivative (tribromo-fluorescein, dianion); eosin derivative (2′,4′,5′,7′-tetrachlor-o-fluorescein, dianion); eosin; eosin dicetylpyridinium chloride ion pair; erythrosin B (2′,4′,5′,7′-tetraiodo-fluorescein, dianion); erythrosin; erythrosin dianion; erythiosin B; fluorescein; fluorescein dianion; phloxin B (2′,4′,5′,7′-tetrabromo-3,4,5,6-tetrachloro-fluorescein, dianion); phloxin B (tetrachloro-tetrabromo-fluorescein); phloxine B; rose bengal (3,4,5,6-tetrachloro-2′,4′,5′,7′-tetraiodofluorescein, dianion); pyronin G, pyronin J, pyronin Y; Rhodamine dyes such as rhodamines include 4,5-dibromo-rhodamine methyl ester; 4,5-dibromo-rhodamine n-butyl ester; rhodamine 101 methyl ester; rhodamine 123; rhodamine 6G; rhodamine 6G hexyl ester; tetrabromo-rhodamine 123; and tetramethyl-rhodamine ethyl ester.

Methylene blue dyes—Exemplary methylene blue derivatives include but are not limited to 1-methyl methylene blue; 1,9-dimethyl methylene blue; methylene blue; methylene violet; bromomethylene violet; 4-iodomethylene violet; 1,9-dimethyl-3-dimethyl-amino-7-diethyl-amino-phenothiazine; and 1,9-dimethyl-3-diethylamino-7-dibutyl-amino-phenot-hiazine.

Azo dyes—Exemplary azo (or diazo-) dyes include but are not limited to methyl violet, neutral red, para red (pigment red 1), amaranth (Azorubine S), Carmoisine (azorubine, food red 3, acid red 14), allura red AC (FD&C 40), tartrazine (FD&C Yellow 5), orange G (acid orange 10), Ponceau 4R (food red 7), methyl red (acid red 2), and murexide-ammonium purpurate.

In some aspects of the disclosure, the one or more light-absorbing molecule is a photosynthetic organism-derived light-absorbing molecule. Examples of photosynthetic organism-derived light-absorbing molecule include, but are not limited to, aloe-emodin, apigenin, berberine, caffeic acid, caffeine, curcumin, gingerol, hyperforin, hypericin, ellagic acid, lycopene, oleuropein, piperine, resveratrol, sanguinarine, tannic acid, theobromine, zeaxanthin, phloroglucinols, adhyperforin, terpenoids, polyphenols, capsaicin, stilbenoids, flavonoids, catechins, capsaicinoids, alkaloids, quinones, ketides, tannins, antraquinones, iridoids, curcuminoids, furocoumarins, phytosterols, carotenoids, isothiocyanates, ginsenosides, withanolides, and derivatives thereof.

In some aspects of the disclosure, the one or more light-absorbing molecules of the biophotonic silicone membranes disclosed herein can be independently selected from any of Acid black 1, Acid blue 22, Acid blue 93, Acid fuchsin, Acid green, Acid green 1, Acid green 5, Acid magenta, Acid orange 10, Acid red 26, Acid red 29, Acid red 44, Acid red 51, Acid red 66, Acid red 87, Acid red 91, Acid red 92, Acid red 94, Acid red 101, Acid red 103, Acid roseine, Acid rubin, Acid violet 19, Acid yellow 1, Acid yellow 9, Acid yellow 23, Acid yellow 24, Acid yellow 36, Acid yellow 73, Acid yellow S, Acridine orange, Acriflavine, Alcian blue, Alcian yellow, Alcohol soluble eosin, Alizarin, Alizarin blue 2RC, Alizarin carmine, Alizarin cyanin BBS, Alizarol cyanin R, Alizarin red S, Alizarin purpurin, Aluminon, Amido black 10B, Amidoschwarz, Aniline blue WS, Anthracene blue SWR, Auramine O, Azocannine B, Azocarmine G, Azoic diazo 5, Azoic diazo 48, Azure A, Azure B, Azure C, Basic blue 8, Basic blue 9, Basic blue 12, Basic blue 15, Basic blue 17, Basic blue 20, Basic blue 26, Basic brown 1, Basic fuchsin, Basic green 4, Basic orange 14, Basic red 2, Basic red 5, Basic red 9, Basic violet 2, Basic violet 3, Basic violet 4, Basic violet 10, Basic violet 14, Basic yellow 1, Basic yellow 2, Biebrich scarlet, Bismarck brown Y, Brilliant crystal scarlet 6R, Calcium red, Carmine, Carminic acid, Celestine blue B, China blue, Cochineal, Coelestine blue, Chrome violet CG, Chromotrope 2R, Chromoxane cyanin R, Congo corinth, Congo red, Cotton blue, Cotton red, Croceine scarlet, Crocin, Crystal ponceau 6R, Crystal violet, Dahlia, Diamond green B, Direct blue 14, Direct blue 58, Direct red, Direct red 10, Direct red 28, Direct red 80, Direct yellow 7, Eosin B, Eosin Bluish, Eosin, Eosin Y, Eosin yellowish, Eosinol, Erie garnet B, Eriochrome cyanin R, Erythrosin B, Ethyl eosin, Ethyl green, Ethyl violet, Evans blue, Fast blue B, Fast green FCF, Fast red B, Fast yellow, Fluorescein, Food green 3, Gallein, Gallamine blue, Gallocyanin, Gentian violet, Haematein, Haematine, Haematoxylin, Helio fast rubin BBL, Helvetia blue, Hematein, Hematine, Hematoxylin, Hoffman's violet, Imperial red, Indocyanin Green, Ingrain blue, Ingrain blue 1, Ingrain yellow 1, INT, Kermes, Kermesic acid, Kernechtrot,

Lac, Laccaic acid, Lauth's violet, Light green, Lissamine green SF, Luxol fast blue, Magenta 0, Magenta I, Magenta II, Magenta III, Malachite green, Manchester brown, Martius yellow, Merbromin, Mercurochrome, Metanil yellow, Methylene azure A, Methylene azure B, Methylene azure C, Methylene blue, Methyl blue, Methyl green, Methyl violet, Methyl violet B, Methyl violet 10B, Mordant blue 3, Mordant blue 10, Mordant blue 14, Mordant blue 23, Mordant blue 32, Mordant blue 45, Mordant red 3, Mordant red 11, Mordant violet 25, Mordant violet 39 Naphthol blue black, Naphthol green B, Naphthol yellow S, Natural black 1, Natural green 3(chlorophyllin), Natural red, Natural red 3, Natural red 4, Natural red 8, Natural red 16, Natural red 25, Natural red 28, Natural yellow 6, NBT, Neutral red, New fuchsin, Niagara blue 2 5 3B, Night blue, Nitro BT, Nitro blue tetrazolium, Nuclear fast red, Orange G, Orcein,

Pararosanilin, Phloxine B, Picric acid, Ponceau 2R, Ponceau 6R, Ponceau B, Ponceau de Xylidine, Ponceau S, Primula, Purpurin, Pyronin B, phycobilins, Phycocyanins, Phycoerythrins. Phycoerythrincyanin (PEC), Phthalocyanines, Pyronin G, Pyronin Y, Quinine, Rhodamine B, Rosanilin, Rose bengal, Saffron, Safranin O, Scarlet R, Scarlet red, Scharlach R, Shellac, Sirius red F3B, Solochrome cyanin R, Soluble blue, Spirit soluble eosin, Sulfur yellow S, Swiss blue, Tartrazine, Thioflavine S, Thioflavine T, Thionin, Toluidine blue, Toluyline red, Tropaeolin G, Trypaflavine, Trypan blue, Uranin, Victoria blue 4R, Victoria blue B, Victoria green B, Vitamin B, Water blue I, Water soluble eosin, Xylidine ponceau, or Yellowish eosin.

In certain embodiments, the biophotonic silicone membranes of the present disclosure includes any of the light-absorbing molecules listed above, or a combination thereof, so as to provide a synergistic biophotonic effect at the application site.

Without being bound to any particular theory, a synergistic effect of the light-absorbing molecule combinations means that the biophotonic effect is greater than the sum of their individual effects. Advantageously, this may translate to increased reactivity of the biophotonic silicone membrane, faster or improved treatment time. Also, the treatment conditions need not be altered to achieve the same or better treatment results, such as time of exposure to light, power of light source used, and wavelength of light used. In other words, use of synergistic combinations of light-absorbing molecules may allow the same or better treatment without necessitating a longer time of exposure to a light source, a higher power light source or a light source with different wavelengths.

In some embodiments, the composition includes Eosin Y as a first light-absorbing molecule and any one or more of Rose Bengal, Fluorescein, Erythrosine, Phloxine B, chlorophyll as a second light-absorbing molecule. It is believed that these combinations have a synergistic effect as they can transfer energy to one another when activated due in part to overlaps or close proximity of their absorption and emission spectra. This transferred energy is then emitted as fluorescence and/or leads to production of reactive oxygen species. This absorbed and re-emitted light is thought to be transmitted throughout the composition, and also to be transmitted into the site of treatment.

In further embodiments, the biophotonic silicone membrane may include, for example, the following synergistic combinations: Eosin Y and Fluorescein; Fluorescein and Rose Bengal; Erythrosine in combination with Eosin Y, Rose Bengal or Fluorescein; Phloxine B in combination with one or more of Eosin Y, Rose Bengal, Fluorescein and Erythrosine. By means of synergistic effects of the light-absorbing molecule combinations in the biophotonic silicone membrane, light-absorbing molecules which cannot normally be activated by an activating light (such as a blue light from an LED), can be activated through energy transfer from light-absorbing molecules which are activated by the activating light. In this way, the different properties of photoactivated light-absorbing molecules can be harnessed and tailored according to the cosmetic or the medical therapy required. For example, Rose Bengal can generate a high yield of singlet oxygen when activated in the presence of molecular oxygen, however it has a low quantum yield in terms of emitted fluorescent light. Rose Bengal has peak absorption around 540 nm and so can be activated by green light. Eosin Y has a high quantum yield and can be activated by blue light. By combining Rose Bengal with Eosin Y, one obtains a composition which can emit therapeutic fluorescent light and generate singlet oxygen when activated by blue light. In this case, the blue light photoactivates Eosin Y, which transfers some of its energy to Rose Bengal as well as emitting some energy as fluorescence.

In some embodiments, the light-absorbing molecule or light-absorbing molecules are selected such that their emitted fluorescent light, on photoactivation, is within one or more of the green, yellow, orange, red and infrared portions of the electromagnetic spectrum, for example having a peak wavelength within the range of about 490 nm to about 800 nm. In certain embodiments, the emitted fluorescent light has a power density of between 0.005 to about 10 mW/cm², about 0.5 to about 5 mW/cm².

Surfactant Phase

The biophotonic silicone membranes of the present disclosure comprise a surfactant phase. The surfactant may be present in an amount of at least 5%, 10%, 15%, 20%, 25%, or 30% of the total membrane. In certain embodiments, the surfactant phase comprises a block copolymer. The term “block copolymer” as used herein refers to a copolymer comprised of 2 or more blocks (or segments) of different homopolymers. The term homopolymer refers to a polymer comprised of a single monomer. Many variations of block copolymers are possible including simple diblock polymers with an A-B architecture and triblock polymers with A-B-A, B-A-B or A-B-C architectures and more complicated block copolymers are known. In addition, unless otherwise indicated herein, the repetition number and type of the monomers or repeating units constituting the block copolymer are not particularly limited. For example, when one denotes the monomeric repeating units as “a” and “b”, it is meant herein that this copolymer includes not only a random copolymer having the average composition of (a)ₘ(b)ₙ, but also a diblock copolymer of the composition (a)ₘ(b)ₙ, and a triblock copolymer of the composition (a)ₗ(b)ₘ(a)ₙ, or the like. In the formulae above, l, m, and n represent the number of repeating units and are positive numbers.

In certain embodiments of any of the foregoing or following the block copolymer is biocompatible. A polymer is “biocompatible” in that the polymer and degradation products thereof are substantially non-toxic to cells or organisms, including non-carcinogenic and non-immunogenic, and are cleared or otherwise degraded in a biological system, such as an organism (patient) without substantial toxic effect.

In certain embodiments the block copolymer of the surfactant phase is from a group of tri-block copolymers designated Poloxamers. Poloxamers are A-B-A block copolymers in which the A segment is a hydrophilic polyethylene glycol (PEG) homopolymer and the B segment is hydrophobic polypropylene glycol (PPG) homopolymer. PEG is also known as polyethylene oxide (PEO) or polyoxyethylene (POE), depending on its molecular weight. Additionally, PPG is also known as polypropylene oxide (PPO), depending on its molecular weight. Poloxamers are commercially available from BASF Corporation. Poloxamers produce reverse thermal gelatin compositions, i.e., with the characteristic that their viscosity increases with increasing temperature up to a point from which viscosity again decreases. Depending on the relative size of the blocks the copolymer can be a solid, liquid or paste. In certain embodiments of the disclosure, the poloxamer is Pluronic® F127 (also known as Poloxamer 407). In some embodiments, the biophotonic silicone membrane may comprise Pluronic® F127 in the amount of 1-40 wt % of the total membrane. In some embodiments of the biophotonic silicone membrane may comprise 1-5 wt %, 2.5-7.5 wt %, 5-10 wt %, 7.5-12.5 wt %, 10-15 wt %, 12.5-17.5 wt %, 15-20 wt %, 20-25 wt %, 25-30 wt %, 30-35 wt %, 35-40 wt % pluronic. In certain embodiments Pluronic® F127 is present in the amount of 2-8 wt % of the total biophotonic silicone membrane.

In certain embodiments of the disclosure the surfactant phase comprises a block copolymer comprising at least an A-B unit, wherein A is PEG and B is polylactic acid (PLA), or polyglycolic acid (PGA) or poly(lactic-co-glycolic acid) (PLGA) or polycaprolactone (PCL) or polydioxanone (PDO). Since the PEG blocks contribute hydrophilicity to the polymer, increasing the length of the PEG blocks or the total amount of PEG in the polymer will tend to make the polymer more hydrophilic. Depending on the amounts and proportions of the other components of the polymer, the desired overall hydrophilicity, and the nature and chemical functional groups of any light-absorbing molecule that may be included in a formulation of the polymer, a skilled person can readily adjust the length (or MW) of the PEG blocks used and/or the total amount of PEG incorporated into the polymer, in order to obtain a polymer having the desired physical and chemical characteristics. The total amount of PEG in the polymer may be about 80 wt % or less, 75 wt % or less, 70 wt % or less, 65 wt % or less, about 60 wt % or less, about 55 wt % or less, or about 50 wt % or less. In particular embodiments, the total amount of PEG is about 55 wt %, 56 wt %, 57 wt %, 58 wt %, 59 wt %, 60 wt %, 61 wt %, 62 wt %, 63 wt %, 64 wt %, 65 wt %, 66 wt %, 67 wt %, 68 wt %, 69 wt %, or about 70 wt %. Unless otherwise specified, a weight percentage of a particular component of the polymer means that the total weight of the polymer is made up of the specified percentage of monomers of that component. For example, 65 wt % PEG means that 65% of the weight of the polymer is made up of PEG monomers, which monomers are linked into blocks of varying lengths, which blocks are distributed along the length of polymer, including in a random distribution.

The total amount of PPG or PLA or PLGA or PCL or PDO present in the block copolymer may be about 50 wt % or less, about 45 wt % or less, about 40 wt % or less, about 35 wt % or less, about 30 wt % or less, about 25 wt % or less, or about 20 wt % or less.

The surfactant phase may also include thickening agents or stabilizers such as gelatin and/or modified celluloses such as hydroxyethyl cellulose (HEC) and carboxymethyl cellulose (CMD), and/or polysaccharides such as xanthan gum, guar gum, and/or starches and/or any other thickening agent. In certain embodiments of the disclosure, the stabilizer or thickening agent may comprise gelatin. For example, the surfactant phase may comprise about 0-5 wt %, about 5-25 wt %, about 0-15 wt %, or about 10-20 wt % gelatin.

Surfactants and/or stabilizers may be selected according to effects they will have on the optical transparency of the biophotonic membrane. The biophotonic silicone membrane should be able to transmit sufficient light to activate the at least one light-absorbing molecule and, in embodiments where fluorescence is emitted by the activated light-absorbing molecule, the surfactant phase should also be able to transmit the emitted fluorescent light to tissues.

Silicone Phase

The biophotonic silicone membranes of the present disclosure comprise a continuous phase of silicone. Silicones are synthetic polymers containing chains consisting of (—Si—O—) repeating unit with two organic groups attached directly to the Si atom.

In certain embodiments, the silicone phase of the biophotonic silicone membrane can be prepared by using commercial kits such as MED-4011, MED-6015, and/or MED-6350 provided by NuSil™. The kit consists in two-part liquid components, the base (part A) and the curing agent or catalyst (part B), both based on polydimethylsiloxane. When mixed at a ratio of 10(A)/1(B) or 1(A)/1(B) the mixture cures to a flexible and transparent elastomer. MED-6015 (“low consistency silicone”) is a silicone elastomer comprising a polydimethyl siloxane and organically-modified silica. The low consistency silicone is prepared by combining a base (Part A) with a curing agent (Part B). The base contains about >60 wt % dimethylvinyl-terminated dimethyl siloxane, about 30 to 60 wt % dimethylvinylated and trimethylated silica and about 1 to 5 wt % tetra(trimethylsiloxy) silane. The curing agent contains about 40 to 70 wt % dimethyl, methylhydrogen siloxane, about 15 to 40 wt % dimethylvinyl-terminated dimethyl siloxane, about 10 to 30 wt % dimethylvinylated and trimethylated silica and about 1 to 5 wt % tetramethyl tetravinyl cyclotetrasiloxane. In another embodiment, the silicone phase of the biophotonic silicone membrane can be prepared by using the MED-6360 (“soft adhesive silicone”) kit, which allows the preparation of a soft and sticky gel, when the two parts A and B are mixed at the ratio 1(A)/1(B). Parts A and B of the kit contain about 85 to 100 wt % dimethylvinyl-terminated dimethyl siloxane and about 1 to 5 wt % dimethyl, methylhydrogen siloxane. In other embodiments, the biophotonic silicone composition may be prepared in a manner to provide for tunable flexibility were desired, for example a silicone-based biophotonic membrane having tunable flexibility. One means of generating a tunable biophotonic silicone membrane of the present disclosure is by combining different ratios of commercially available PDMS such as MED-4011, MED-6015, and/or MED-6350. In some embodiments the silicone phase comprises MED-6360 in the amount of 5-100 wt % of the silicone phase. In certain embodiments of the present disclosure the MED-6350 is present in an amount of about 5-10 wt %, 10-15 wt %, 15-20 wt %, 20-25 wt %, 25-30 wt %, 30-35 wt %, 35-40 wt %, 40-45 wt %, 45-50 wt %, 50-55 wt %, 55-60 wt %, 60-65 wt % 65-70 wt %, 70-75 wt %, 75-80 wt %, 80-85 wt %, 85-90 wt %, 90-95 wt % or 95-100 wt % of the silicone phase. In certain embodiments of the present disclosure, the silicone phase comprises MED-6015. In certain other embodiments of the present disclosure, the MED-6015 is present in an amount of about 5-10 wt %, 10-15 wt %, 15-20 wt %, 20-25 wt %, 25-30 wt %, 30-35 wt %, 35-40 wt %, 40-45 wt %, 45-50 wt %, 50-55 wt %, 55-60 wt %, 60-65 wt % 65-70 wt %, 70-75 wt %, 75-80 wt %, 80-85 wt %, 85-90 wt %, 90-95 wt % or 95-100 wt % of the silicone phase. In certain other embodiments of the present disclosure, the MED-4011 is present in an amount of about 5-10 wt %, 10-15 wt %, 15-20 wt %, 20-25 wt %, 25-30 wt %, 30-35 wt %, 35-40 wt %, 40-45 wt %, 45-50 wt %, 50-55 wt %, 55-60 wt %, 60-65 wt % 65-70 wt %, 70-75 wt %, 75-80 wt %, 80-85 wt %, 85-90 wt %, 90-95 wt % or 95-100 wt % of the silicone phase.

In one embodiment, the silicone phase of the biophotonic silicone membrane is a mixture using 70% MED-6360 and 30% of either MED-4011 or MED-6015. The MED-4011 kit produces a “low consistency silicone”. The components A and B of MED-4011 have well defined properties. For example, the viscosity of component A and component B, uncured, is 105,000 mPas and 1,500 mPas, respectively. Components A and B mixed at a ratio of 10/1 generates the low consistency silicone elastomer with tensile strength of 670 psi, post-cured. As used herein, “low consistency silicone” is understood to refer to a silicone composition produced by the MED-4011 kit. These terms (“low consistency silicone” and “MED-4011”) are sometimes used interchangeably. The MED-6015 kit produces a “clear low consistency silicone”. The components A and B of MED-6015 have well defined properties. For example, the viscosity of component A and component B, uncured, is 5,500 mPas and 95 mPas, respectively. Components A and B mixed at a ratio of 10/1 generates the clear low consistency silicone elastomer with tensile strength of 1200 psi, post-cured. As used herein, “clear low consistency silicone” is understood to refer to a silicone composition produced by the MED-6015 kit. These terms (“clear low consistency silicone” and “MED-6015”) are sometimes used interchangeably.

The MED-6350 kit produces a “soft adhesive silicone”. The components A and B of MED-6350 have well defined properties. For example, the viscosity of component A and component B, uncured, is 25,000 mPas and 16,500 mPas, respectively. Components A and B mixed at a ratio of 1/1 generates the soft adhesive silicone with a surface tack measurement of 5.7 psi, post-cured. As used herein, “soft adhesive silicone” is understood to refer to a silicone composition produced by the MED-6350 kit. These terms (“sot adhesive silicone” and “MED-6350”) are sometimes used interchangeably.

In one embodiment, the silicone phase of the biophotonic silicone membrane is a mixture using MED-4011 or MED-6015 with MED-6360 at the following ratios: 10/90, 20/80, 30/70, 40/60, 50/50, 60/40, 70/30, 80/20, or 90/10. For example, in one embodiment, the silicone phase of the biophotonic silicone membrane is a mixture using 30% MED-4011 or 30% MED-6015 with 70% MED-6360 (i.e., 30/70). In certain embodiments, the biophotonic silicone membrane can also comprise a thin outer coating comprising of MED-6360 (e.g., part A and part B mixed at 1:1) for enhanced adhesiveness. In some embodiments, the outer coating has a thickness in a range of about 50 μm to about 500 μm. In some embodiments, the outer coating has a thickness in a range of about 50 μm to about 75 μm, about 75 μm to about 100 μm, about 100 μm to about 125 μm, about 125 μm to about 150 μm, about 150 μm to about 175 μm, about 175 μm to about 200 μm, about 200 μm to about 225 μm, about 225 μm to about 250 μm, about 250 μm to about 275 μm, 275 μm to about 300 μm, about 300 μm to about 325 μm, about 325 μm to about 350 μm, about 350 μm to about 375 μm, about 375 μm to about 400 μm, about 400 μm to about 425 μm, about 425 μm to about 450 μm, about 450 μm to about 475 μm, or about 475 μm to about 500 μm thick.

In certain embodiments, the silicone is not a polydimethylsiloxane (PDMS) fluid (Me₂SiO)ₙ or a PDMS-based gel or PDMS-based elastomer.

Optical Properties of the Biophotonic Silicone Membranes

In certain embodiments, biophotonic silicone compositions of the present disclosure are substantially transparent or translucent. The % transmittance of the biophotonic silicone membrane can be measured in the range of wavelengths from 250 nm to 800 nm using, for example, a Perkin-Elmer Lambda 9500 series UV-visible spectrophotometer. In some embodiments, transmittance within the visible range is measured and averaged. In some other embodiments, transmittance of the biophotonic silicone membrane is measured with the light-absorbing molecule omitted. As transmittance is dependent upon thickness, the thickness of each sample can be measured with calipers prior to loading in the spectrophotometer. In some embodiments, the biophotonic silicone membrane has a transmittance that is more than about 20%, 30%, 40%, 50%, 60%, 70%, or 75% within the visible range. In some embodiments, the transmittance exceeds 40%, 41%, 42%, 43%, 44%, or 45% within the visible range. In some embodiments, the biophotonic silicone membrane has a light transmittance of about 40-100%, 45-100%, 50-100%, 55-100%, 60-100%, 65-100%, 70-100%, 75-100%, 80-100%, 85-100%, 90-100%, or 95-100%.

Forms of the Biophotonic Silicone Membranes

The biophotonic silicone membranes of the present disclosure may be deformable. They may be elastic or non-elastic (i.e. flexible or rigid). The biophotonic silicone membrane, for example, may be in a peel-off form(‘peelable’) to provide ease and speed of use. In certain embodiments, the tear strength and/or tensile strength of the peel-off form is greater than its adhesion strength. This may help handleability of the biophotonic silicone membrane. It will be recognized by one of skill in the art that the properties of the peel-off biophotonic silicone membrane such as cohesiveness, flexibility, elasticity, tensile strength, and tearing strength, can be determined and/or adjusted by methods known in the art such as by selecting suitable PDMS-based compositions and adapting their relative ratios. The biophotonic silicone membrane may be provided in a pre-formed shape. In certain embodiments, the pre-formed shape is in the form of, including, but not limited to, a film, a face mask, a patch, a dressing, or bandage. In certain embodiments, the pre-formed shapes can be customized for the individual user by trimming to size. In certain embodiments, perforations are provided around the perimeter of the pre-formed shape to facilitate trimming In certain embodiments, the pre-shaping can be performed manually or by mechanical means such as 3-D printing. In the case of the 3-D printing the size of the area to be treated can be imaged, such as a post-surgical area or a face, then a 3-D printer configured to build or form a cohesive biophotonic silicone membrane to match the size and shape of the imaged treatment area.

A biophotonic silicone membrane of the disclosure can be configured with a shape and/or size for application to a desired portion of a subject's body. For example, the biophotonic silicone membrane can be shaped and sized to correspond with a desired portion of the body to receive the biophotonic treatment. Such a desired portion of skin can be selected from, but not limited to, the group consisting of a skin, head, forehead, scalp, nose, cheeks, lips, ears, face, neck, shoulder, arm pit, arm, elbow, hand, finger, abdomen, chest, breast, stomach, back, buttocks, sacrum, genitals, legs, knee, feet, toes, nails, hair, any boney prominences, and combinations thereof, and the like. Thus, the biophotonic silicone membrane of the disclosure can be shaped and sized to be applied to any portion of skin on a subject's body. For example, the biophotonic silicone membrane can be in the form of a sock, hat, glove or mitten shaped form. In embodiments where the biophotonic silicone membrane is in an elastic, semi-rigid or rigid form, it may be peeled-off without leaving any residue on the tissue.

In certain embodiments, the biophotonic silicone membrane is provided in the form of an elastic and peelable face mask, which may be pre-formed. In other embodiments, the biophotonic silicone membrane is in the form of a non-elastic (rigid) face mask, which may also be pre-formed. The mask can have openings for one or more of the eyes, nose and mouth. In a further embodiment, the openings are protected with a covering, or the exposed skin such as on the nose, lips or eyes are protected using for example cocoa butter. In certain embodiments, the pre-formed face mask is provided in the form of multiple parts, e.g., an upper face part and a lower face part. In certain embodiments, the uneven proximity of the face to a light source is compensated for, e.g., by adjusting the thickness of the mask, or by adjusting the amount of light-absorbing molecule in the different areas of the mask, or by blocking the skin in closest proximity to the light. In certain embodiments, the pre-formed shapes come in a one-size fits all form.

In certain embodiments, the biophotonic silicone membrane is in the form of a dressing or a bandage. It may be used on a post-surgical area to prevent or limit scar formation, or on an existing scar to diminish the appearance of the scar.

In certain aspects, the mask (or patch) is not pre-formed and is applied e.g., by spreading a biophotonic silicone membrane making up the mask (or patch), on the skin or target tissue, or alternatively by smearing, dabbing or rolling the composition on target tissue. It can then be converted to a peel-off form after application, by means such as, but not limited to, drying or inducing a change in temperature upon application to the skin or tissue. After use, the mask (or patch) can then be peeled off without leaving any flakes on the skin or tissue, preferably without wiping or washing.

The biophotonic silicone membranes of the present disclosure may have a thickness of from about 0.1 mm to about 50 mm, about 0.5 mm to about 20 mm, or about 1 mm to about 10 mm. It will be appreciated that the thickness will vary based on the intended use. In some embodiments, the thickness ranges from about 0.1-1 mm. In some embodiments, the thickness ranges from about 0.5-1.5 mm, about 1-2 mm, about 1.5-2.5 mm, about 2-3 mm, about 2.5-3.5 mm, about 3-4 mm, about 3.5-4.5 mm, about 4-5 mm, about 4.5-5.5 mm, about 5-6 mm, about 5.5-6.5 mm, about 6-7 mm, about 6.5-7.5 mm, about 7-8 mm, about 7.5-8.5 mm, about 8-9 mm, about 8.5-9.5 mm, about 9-10 mm, about 10-11mm, about 11-12 mm, about 12-13 mm, about 13-14 mm, about 14-15 mm, about 15-16 mm, about 16-17 mm, about 17-18 mm, about 18-19 mm, about 19-20 mm, about 20-22 mm, about 22-24 mm, about 24-26 mm, about 26-28 mm, about 28-30 mm, about 30-35 mm, about 35-40 mm, about 40-45 mm, about 45-50 mm.

The tensile strength of the biophotonic silicone membranes will vary based on the intended use. The tensile strength can be determined by performing a tensile test and recording the force and displacement. These are then converted to stress (using cross sectional area) and strain; the highest point of the stress-strain curve is the “ultimate tensile strength.” In some embodiments, for example when in the form of a biophotonic silicone membrane, tensile strength can be characterized using a 500N capacity tabletop mechanical testing system (#5942R4910, Instron®) with a 5N maximum static load cell (#102608, Instron). Pneumatic side action grips can be used to secure the samples (#2712-019, Instron). In some embodiments, a constant extension rate (for example, of about 2 mm/min) until failure can be applied and the tensile strength is calculated from the stress vs. strain data plots. In some embodiments, the tensile strength can be measured using methods as described in or equivalent to those described in American Society for Testing and Materials tensile testing methods such as ASTM D638, ASTM D882 and ASTM D412.

In some embodiments, the biophotonic silicone membrane has a tensile strength that is at least about 50 kPa, at least about 100 kPa, at least about 200 kPa, at least about 300 kPa, at least about 400 kPa, at least about 500 kPa, at least about 600 kPa, at least about 700 kPa, at least about 800 kPa, at least about 900 kPa, at least about 1 MPa, at least about 2 MPa or at least about 3 MPa, or at least about 5 MPa, or at least about 6 MPa. In some embodiments, the tensile strength of the biophotonic silicone membrane is up to about 10 MPa.

The tear strength of the biophotonic silicone composition will vary depending on the intended use. The tear strength property of the biophotonic silicone membrane can be tested using a 500N capacity tabletop mechanical testing system (#5942R4910, Instron) with a 5N maximum static load cell (#102608, Instron). Pneumatic side action grips can be used to secure the samples (#2712-019, Instron). Samples can be tested with a constant extension rate (for example, of about 2 mm/min) until failure. In accordance with the technology, tear strength is calculated as the force at failure divided by the average thickness (N/mm).

In some embodiments, the biophotonic silicone membrane has a tear strength of from about 0.1 N/mm to about 5 N/mm. In some embodiments, the tear strength is from about 0.1 N/mm to about 0.5 N/mm, from about 0.25 N/mm to about 0.75 N/mm, from about 0. 5 N/mm to about 1.0 N/mm, from about 0.75 N/mm to about 1.25 N/mm, from about 1.0 N/mm to about 1.5 N/mm, from about 1.5 N/mm to about 2.0 N/mm, from about 2.0 N/mm to about 2.5 N/mm, from about 2.5 N/mm to about 3.0 N/mm, from about 3.0 N/mm to about 3.5 N/mm, from about 3.5 N/mm to about 4.0 N/mm, from about 4.0 N/mm to about 4.5 N/mm, from about 4.5 N/mm to about 5.0 N/mm.

The adhesion strength of the biophotonic silicone membrane will vary depending on the intended use. Adhesion strength can be determined in accordance with ASTM D-3330-78, PSTC-101 and is a measure of the force required to remove a biophotonic silicone membrane from a test panel at a specific angle and rate of removal. In some embodiments, a predetermined size of the biophotonic silicone membrane is applied to a horizontal surface of a clean glass test plate. A hard rubber roller is used to firmly apply a piece of the biophotonic silicone membrane and remove all discontinuities and entrapped air. The free end of the piece of biophotonic silicone membrane is then doubled back nearly touching itself so that the angle of removal of the piece from the glass plate will be 180 degrees. The free end of the piece of biophotonic silicone membrane is attached to the adhesion tester scale (e.g. an Instron tensile tester or Harvey tensile tester). The test plate is then clamped in the jaws of the tensile testing machine capable of moving the plate away from the scale at a predetermined constant rate. The scale reading in kg is recorded as the biophotonic silicone membrane is peeled from the glass surface.

In some embodiments, the adhesion strength can be measured by taking into account the static friction of the biophotonic silicone membrane. For some embodiments of the biophotonic silicone membranes of the present disclosure, the adhesive properties are linked to their levels of static friction, or stiction. In these cases, the adhesion strength can be measured by placing a sample of the biophotonic silicone membrane on a test surface and pulling one end of the sample at an angle of approximately 0° (substantially parallel to the surface) whilst applying a known downward force (e.g. a weight) on the sample and measuring the weight at which the sample slips from the surface. The normal force Fₙ, is the force exerted by each surface on the other in a perpendicular (normal) direction to the surface and is calculated by multiplying the combined weight of the sample and the weight by the gravity constant (g) (9.8 m/s²). The sample with the weight on top is then pulled away from a balance until the sample slips from the surface and the weight is recorded on the scale. The weight recorded on the scale is equivalent to the force required to overcome the friction. The force of friction (F_{f}) is then calculated by multiplying the weight recorded on the scale by g. Since F_{f}≤μFₙ (Coulomb's friction law), the friction coefficient μ can be obtained by dividing F_{f}/Fₙ. The stress required to shear a material from a surface (adhesion strength) can then be calculated from the friction coefficient, μ, by multiplying the weight of the material by the friction coefficient.

In some embodiments, the biophotonic silicone membrane has an adhesion strength that is less than its tensile strength or its tear strength.

In some embodiments, the biophotonic silicone membrane has adhesion strength of from about 0.01 N/mm to about 0.60 N/mm. In some embodiments, the adhesion strength is from about 0.20 N/mm to about 0.40 N/mm, or from about 0.25 N/mm to about 0.35 N/mm. In some embodiments, the adhesion strength is less than about 0.10 N/mm, less than about 0.15 N/mm, less than about 0.20 N/mm, less than about 0.25 N/mm, less than about 0.30 N/mm, less than about 0.35 N/mm, less than about 0.40 N/mm, less than about 0.45 N/mm, less than about 0.55 N/mm or less than about 0.60 N/mm.

Methods of Use

The biophotonic silicone membranes of the present disclosure may have cosmetic and/or medical benefits. In certain embodiments, the present disclosure provides a method for preventing or treating scarring, the method comprising: applying a biophotonic silicone membrane of the present disclosure to the area of the skin or tissue in need of treatment, and illuminating the biophotonic silicone membrane with light having a wavelength that overlaps with an absorption spectrum of the light-absorbing molecule(s) present in the membrane. In certain embodiments, the biophotonic silicone membrane of the present disclosure is used to prevent or treat scars, including but not limited to linear scars, hypertrophic scars, keloid scars, sunken scars, and stretched scars. The scar to be prevented or treated can result from a number of causes, including but not limited to injury or surgery. In one embodiment, the scar to be prevented or treated is a post-surgical scar resulting from, e.g., bilateral breast reduction.

In the methods of the present disclosure, any source of actinic light can be used. Any type of halogen, LED or plasma arc lamp, or laser may be suitable. The primary characteristic of suitable sources of actinic light will be that they emit light in a wavelength (or wavelengths) appropriate for activating the one or more photoactivators present in the composition. In one embodiment, an argon laser is used. In another embodiment, a potassium-titanyl phosphate (KTP) laser (e.g. a GreenLight™ laser) is used. In yet another embodiment, a LED lamp such as a photocuring device is the source of the actinic light. In yet another embodiment, the source of the actinic light is a source of light having a wavelength between about 200 to 800 nm. In another embodiment, the source of the actinic light is a source of visible light having a wavelength between about 400 and 600 nm. In another embodiment, the source of the actinic light is a source of visible light having a wavelength between about 400 and 700 nm. In yet another embodiment, the source of the actinic light is blue light. In yet another embodiment, the source of the actinic light is red light. In yet another embodiment, the source of the actinic light is green light. Furthermore, the source of actinic light should have a suitable power density. Suitable power density for non-collimated light sources (LED, halogen or plasma lamps) are in the range from about 0.1 mW/cm² to about 200 mW/cm². Suitable power density for laser light sources are in the range from about 0.5 mW/cm² to about 0.8 mW/cm².

In some embodiments of the methods of the present disclosure, the light has an energy at the subject's skin surface of between about 0.1 mW/cm² and about 500 mW/cm², or 0.1-300 mW/cm², or 0.1-200 mW/cm², wherein the energy applied depends at least on the condition being treated, the wavelength of the light, the distance of the skin from the light source and the thickness of the biophotonic material. In certain embodiments, the light at the subject's skin is between about 1-40 mW/cm², or 20-60 mW/cm², or 40-80 mW/cm², or 60-100 mW/cm², or 80-120 mW/cm², or 100-140 mW/cm², or 30-180 mW/cm², or 120-160 mW/cm², or 140-180 mW/cm², or 160-200 mW/cm², or 110-240 mW/cm², or 110-150 mW/cm², or 190-240 mW/cm².

The activation of the light-absorbing molecule(s) within the biophotonic silicone membrane may take place almost immediately on illumination (femto- or pico seconds). A prolonged exposure period may be beneficial to exploit the synergistic effects of the absorbed, reflected and reemitted light of the biophotonic silicone membrane of the present disclosure and its interaction with the tissue being treated. In one embodiment, the time of exposure of the tissue or skin or biophotonic silicone membrane to actinic light is a period between 0.01 minutes and 90 minutes. In another embodiment, the time of exposure of the tissue or skin or biophotonic silicone membrane to actinic light is a period between 1 minute and 5 minutes. In some other embodiments, the biophotonic silicone membrane is illuminated for a period between 1 minute and 3 minutes. In certain embodiments, light is applied for a period of 1-30 seconds, 15-45 seconds, 30-60 seconds, 0.75-1.5 minutes, 1-2 minutes, 1.5-2.5 minutes, 2-3 minutes, 2.5-3.5 minutes, 3-4 minutes, 3.5-4.5 minutes, 4-5 minutes, 5-10 minutes, 10-15 minutes, 15-20 minutes, or 20-30 minutes. The treatment time may range up to about 90 minutes, about 80 minutes, about 70 minutes, about 60 minutes, about 50 minutes, about 40 minutes or about 30 minutes. It will be appreciated that the treatment time can be adjusted in order to maintain a dosage by adjusting the rate of fluence delivered to a treatment area. For example, the delivered fluence may be about 4 to about 60 J/cm², about 10 to about 60 J/cm², about 10 to about 50 J/cm², about 10 to about 40 J/cm², about 10 to about 30 J/cm², about 20 to about 40 J/cm², about 15 J/cm² to 25 J/cm², or about 10 to about 20 J/cm².

In certain embodiments, the biophotonic silicone membrane may be re-illuminated at certain intervals. In yet another embodiment, the source of actinic light is in continuous motion over the treated area for the appropriate time of exposure. In yet another embodiment, the biophotonic silicone membrane may be illuminated until the biophotonic silicone membrane is at least partially photobleached or fully photobleached.

In certain embodiments, the light-absorbing molecule(s) may be photoexcited by ambient light including from the sun and overhead lighting. In certain embodiments, the light-absorbing molecule(s) may be photoactivated by light in the visible range of the electromagnetic spectrum. The light may be emitted by any light source such as sunlight, light bulb, an LED device, electronic display screens such as on a television, computer, telephone, mobile device, flashlights on mobile devices. In the methods of the present disclosure, any source of light can be used. For example, a combination of ambient light and direct sunlight or direct artificial light may be used. Ambient light can include overhead lighting such as LED bulbs, fluorescent bulbs etc, and indirect sunlight.

In the methods of the present disclosure, the biophotonic silicone membrane may be removed from the skin following application of light. In other embodiments, the biophotonic silicone membrane is left on the tissue for an extended period of time and re-activated with direct or ambient light at appropriate times to treat the condition.

In certain embodiments of any of the foregoing or following, the biophotonic silicone membrane has a removable cover for covering one or both sides of the membrane. The removable cover may be peelable. The removable cover may comprise a sheet or a film of material, such as paper or foil. In certain embodiments, the removable cover is opaque and can protect the membrane from illumination until the treatment time. The cover may be partially removable. In certain embodiments, the cover may be re-applicable to the membrane surface, such as after a treatment time, in order to protect the membrane from further illumination in between treatments.

In certain embodiments of the method of the present disclosure, the biophotonic silicone membrane may be applied to the tissue, such as on the face, once, twice, three times, four times, five times or six times a week, daily, or at any other frequency. The total treatment time may be one week, two weeks, three weeks, four weeks, five weeks, six weeks, seven weeks, eight weeks, nine weeks, ten weeks, eleven weeks, twelve weeks, or any other length of time deemed appropriate. In certain embodiments, the total tissue area to be treated may be split into separate areas (cheeks, forehead, breast), and each area treated separately. For example, the biophotonic silicone membrane may be applied topically to a first portion, and that portion illuminated with light, and the composition then removed. Then the biophotonic silicone membrane is applied to a second portion, illuminated and removed. Finally, the biophotonic silicone membrane is applied to a third portion, illuminated and removed.

In certain embodiments, the biophotonic silicone membrane can be used following a surgical procedure to optimize scar revision. In this case, the biophotonic silicone membrane may be applied at regular intervals such as once a week, or at an interval deemed appropriate by the physician.

In the methods of the present disclosure, additional components may optionally be included with the biophotonic silicone membrane or used in combination with the biophotonic silicone membranes. Such additional components may include, but are not limited to, healing factors, antimicrobials, oxygen-rich agents, wrinkle fillers such as botox, hyaluronic acid and polylactic acid, fungal, anti-bacterial, anti-viral agents and/or agents that promote collagen synthesis. Agents that promote collagen synthesis (i.e., pro-collagen synthesis agents) include amino acids, peptides, proteins, lipids, small chemical molecules, natural products and extracts from natural products. These additional components may be applied to the skin in a topical fashion, prior to, at the same time of, and/or after topical application of the biophotonic silicone membranes of the present disclosure. Suitable healing factors comprise compounds that promote or enhance the healing or regenerative process of the tissues on the application site. During the photoactivation of a biophotonic silicone membrane of the present disclosure, there may be an increase of the absorption of molecules of such additional components at the treatment site by the skin or the mucosa. Healing factors may also modulate the biophotonic effect resulting from the biophotonic silicone membrane. Suitable healing factors include, but are not limited to glucosamines, allantoins, saffron, agents that promote collagen synthesis, anti-fungal, anti-bacterial, anti-viral agents and wound healing factors such as growth factors.

Kits

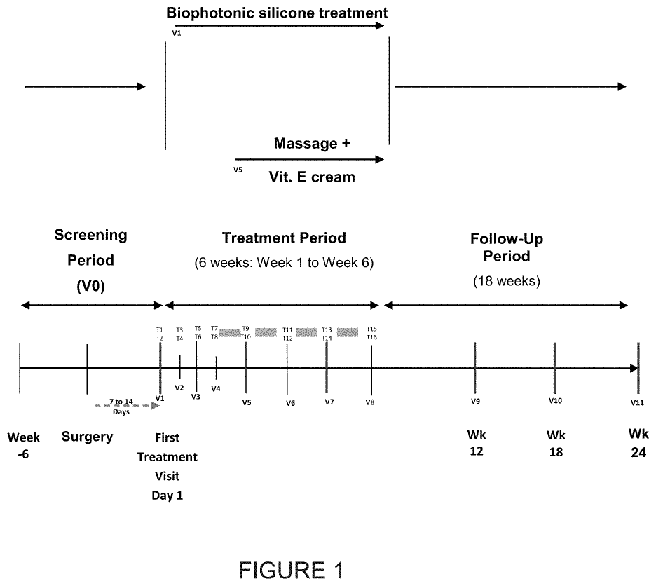

The present disclosure also provides a kit comprising a biophotonic silicone membrane described here (e.g., having a silicone phase and a surfactant phase, and wherein the surfactant phase comprises at least one light-absorbing molecule solubilized in a surfactant); and instructions for performing any of the methods described herein, e.g., the methods as provided in Example 3. For example, the kit comprises instructions to apply the non-adherent side of the biophotonic silicone membrane on the wound at treatment visits 1, 2, and 3 (see FIG. 1), and to apply the adherent side of the biophotonic silicone membrane at treatment visits 4, 5, 6, 7, and 8 (see FIG. 1). During treatment visits 1-8, the site being treated with the biophotonic silicone membrane is to be illuminated two consecutive times for 5 minutes for a total of 10 minutes with a break period (no illumination) of 1 to 2 minutes between illuminations. The multi-LED lamp is positioned such that the illumination is performed at a distance of 5 cm from the site. The same biophotonic silicone membrane is used for the two illuminations. In some embodiments, the kit also comprises a multi-LED lamp.

The present disclosure also provides kits for preparing a biophotonic silicone membranes and/or providing any of the components required for forming biophotonic silicone membranes of the present disclosure. In some embodiments, the kit includes containers comprising the components or compositions that can be used to make the biophotonic silicone membranes of the present disclosure. In some embodiments, the kit includes the biophotonic silicone membrane of the present disclosure. The different components making up the biophotonic silicone membranes of the present disclosure may be provided in separate containers. For example, the surfactant phase may be provided in a container separate from the silicone phase. Examples of such containers are dual chamber syringes, dual chamber containers with removable partitions, sachets with pouches, and multiple-compartment blister packs. Another example is one of the components being provided in a syringe which can be injected into a container of another component. In other embodiments, the kit comprises a systemic drug for augmenting the treatment of the biophotonic silicone membrane of the present disclosure. For example, the kit may include a systemic or topical antibiotic, hormone treatment, or a negative pressure device. In other embodiments, the kit comprises a means for mixing or applying the components of the biophotonic silicone membranes. In certain embodiments of the kit, the kit may further comprise a light source such as a portable light with a wavelength appropriate to activate the light-absorbing molecule of the biophotonic silicone membrane. The portable light may be battery operated or re-chargeable. Written instructions on how to use the biophotonic silicone membranes in accordance with the present disclosure may be included in the kit, or may be included on or associated with the containers comprising the biophotonic silicone membrane or the components making up the biophotonic silicone membranes of the present disclosure. Identification of equivalent biophotonic silicone membranes, methods and kits are well within the skill of the ordinary practitioner and would require no more than routine experimentation, in light of the teachings of the present disclosure.

Variations and modifications will occur to those of skill in the art after reviewing this disclosure. The disclosed features may be implemented, in any combination and sub-combinations (including multiple dependent combinations and sub-combinations), with one or more other features described herein. The various features described or illustrated above, including any components thereof, may be combined or integrated in other systems. Moreover, certain features may be omitted or not implemented. Examples of changes, substitutions, and alterations are ascertainable by one skilled in the art and could be made without departing from the scope of the information disclosed herein. All references cited herein are incorporated by reference in their entirety and made part of this application. Practice of the disclosure will be still more fully understood from the following examples, which are presented herein for illustration only and should not be construed as limiting the disclosure in any way.

### EXAMPLES

The present study compares the safety and efficacy of the biophotonic silicone membrane in the treatment of newly formed post-surgical scars to standard of care. The efficacy of the biophotonic silicone membrane in the reduction of the risk of developing hypertrophic scars and keloids on post-surgical wounds was examined.

### Example 1: Preparation of Biophotonic Silicone Membrane

A biophotonic silicone membrane was prepared by using a commercial Silicone Elastomer kit. The kit comprised two-part viscous liquid components, the base (part A) and the curing agent or catalyst (part B), both based on polydimethylsiloxane (PDMS). For the present study, medical grade silicone kits MED-4011, MED-6015, and MED-6360, provided by NuSil were selected. For MED-4011 and MED-6015 kits, when the parts A & B are mixed at a ratio 10(A)/1(B), the mixtures cure to flexible and transparent elastomers. Although both elastomers seem have the same appearance, they differ by their mechanical properties, as the tensile strength of the elastomer from MED-4011 is much higher due to the length of polydimethylsiloxane chains, which are much longer in MED-4011 than in MED-6015. For the MED-6360 kit, when the two parts A & B are mixed at the ratio 1/1, a very soft and sticky gel is produced upon curing. To obtain membranes with tunable tensile strength and flexibility a mixture of kits were used. Thus, mixtures of either 30% of MED-4011 or 30% of MED-6015 with 70% MED-6360 have been found the most appropriate for the present method. Typical preparations of these mixtures are detailed herein. MED-4011/MED-6360-Silicone mixture of MED-4011 (30%) and MED-6360 (70%) was prepared by thoroughly mixing 3.611 g of MED-4011, composed of 3.277 g of part A and 0.334 g of part B, with 8.418 g of MED-6360, composed of 4.203 g of part A and 4.215 g of part B. MED-6015/MED-6360-Silicone mixture MED-6015 (30%) and MED-6360 (70%) was prepared by thoroughly mixing 3.607 g of MED-6015, composed of 3.277 g of part A and 0.330 g of part B, with 8.408 g of MED-6360, composed of 4.203 g of part A and 4.205 g of part B. Once prepared, these mixtures can be kept much longer in liquid form when cooled to about 4° C.

Preparation of biophotonic silicone membrane—The biophotonic silicone membrane including a silicone matrix containing 15 to 30% of the aqueous phase (Thermogel/TEA/Eosin). 3.0 mL of cold Pluronic-F127 themogelling solution containing light-absorbing molecules were added to 7.020 g of freshly prepared Silicone mixture, MED-4011/MED-6360 or MED-6015/MED-6360, under vigorous stirring to create an extremely fine emulsion. Then, the resulting mixture was casted onto petri dishes. The casted amount allowed the control of the membrane thickness, which is preferentially between 1 and 2 mm. The petri dishes were then cured for 24 hours at 40° C. and under saturated humid atmosphere in an incubator. The resulting biophotonic membrane contained 30% of aqueous phase. The resulting membranes appeared uniform, showing desired flexibility and wrapping intimately the fine microgelled droplets containing light-absorbing molecules. This prevents the leaching of both the Pluronic-F127 gel and the light-absorbing molecules as has been observed after immersion in PBS solution during 24 hours.

Preparation of biophotonic adhesive silicone membrane—the biophotonic silicone membrane was prepared as described above. Thereafter, the membrane was removed from the incubator and coated with very thin layer of silicone MED-6360 (part A and part B mixed at a ratio 1/1), then returned in the incubator for an additional 16 hours of curing. This extra, outer thin coating of MED-6360 (mixture of part A and part B at a ratio 1/1) is expected to intimately integrate to silicone membrane and confer to it the desired adhesiveness (i.e., stickiness). This thin layer is expected to intimately integrate to silicone membrane and make it sticky as MED-6360 (mixture of part A and part B at a ratio 1/1) is known to give sticky gel upon curing. Any silicone known to give sticky elastomer gel upon curing can be used.

### Example 2: Biophotonic Silicone Membrane Promote Wound Closure

The biophotonic silicone membrane was prepared as described above. Briefly, the biophotonic silicone membrane was produced with MED-4011 and MED-6360 from NuSil Technology, which are both high purity Medical grade elastomers. The photoconverting ingredient/molecule (light-absorbing molecules), were first dissolved in a self-gelling polymer aqueous solution, which in turn is homogeneously dispersed as a fine emulsion within a silicone matrix. The silicone matrix was then formed into a thin 1.0 mm sheet through a knife coating process. It was then vulcanized to permanently entrap the photoconverting molecules within the silicone matrix and fully isolate it from skin or injured tissues during treatment. The sheet was then cut, packaged and terminally sterilized by autoclaving. Each biophotonic silicone membrane was sealed in a breathable sterilization pouch as a bacterial barrier, and individually inserted in a sealed aluminium foil pouch to provide protection from light and environmental conditions. Each biophotonic silicone membrane had an adherent and a non-adherent side. The adherent side was attached to the transparent side of the primary packaging, whereas the non-adherent side was attached to the non-transparent side of the primary packaging. The sterile single-use biophotonic silicone membrane was applied to the treatment area(s), and illuminated for a predetermined period of time using a multi-LED lamp. In some embodiments, the multi-LED lamp device delivered non-coherent blue light with a peak wavelength in the range of 440 to 460 nm having a power density of about 50 to 150 mW/cm² at a distance of 5 cm from the light. The dimensions of each biophotonic silicone membrane were about 7 cm by about 11 cm. The biophotonic silicone membrane was tested in vitro on Dermal Human Fibroblasts (DHF) cultures to assess the effect of the treatment on the secretion of inflammatory mediators, growth factors, and tissue remodeling proteins. It was also evaluated in vivo on a human/mouse hypertrophic scar model. Seven days following grafting the mice were treated twice a week for six (6) weeks with a Silicone-Membrane in combination with a multi-LED lamp (as described herein) placed at a 5-cm distance from the graft. Graft biopsies were analyzed for dermis thickness, collagen, and presence of myofibroblasts, mast cells, macrophages, vascularity and CTFG production. The treatment significantly decreased PDGF-BB, TGFb1 and CTGF, three important growth factors implicated in the pathogenesis of scarring. The treatment also inhibited TGFβ1-induced collagen synthesis in dermal human fibroblasts (characteristic of hypertrophic scar formation). The treatment stimulated collagen remodelling, as noted by a very significant decrease in Collagen Orientation Index against untreated control, returning close to normal skin value within three (3) months. Also, the treatment resulted in a decrease in myofibroblast population significantly faster than in untreated control (myofibroblasts are important factor in hypertrophic scar development).

### Example 3: Scar Treatment—Study Design, Study Procedures and Treatment

A study was performed with 5 patients having bilateral breast reduction. The two breasts of each patient were randomized in one of the two following treatment options:
a. One breast treated with the biophotonic silicone membrane as defined in Example 2 and illuminated by the multi-LED lamp as described herein;
b. The second breast treated with Standard of Care only, consisting on wound massages with Vitamin E cream;
c. The two breasts assessed for Safety and Efficacy criteria.

Patients were seen three times during the Follow-up period, at Weeks 12, 18 and 24 after the start of treatment. POSAS and VSS scales were used at different time points during the study. External, surface echography measurements of the scar will be realized at specific Treatment and Follow-up visits. An overview of the Study Design is illustrated in FIG. 1.

The treatment period started 7 days after the surgery, with an authorized visit window of +7 days, meaning that the treatment had to start maximum 14 days post-surgery. No treatment was performed if there were visible sutures on the wound/scar. The breasts treated with the biophotonic silicone membrane or with Standard of care (massages with Vitamin E cream) were randomly selected (“Left” and “Right” breast). During the treatment period, patients were seen twice-weekly during the first two weeks of treatment and then once a week for the four other weeks of treatment. Eight treatments visits are planned in total. Once the treatment period with the biophotonic silicone membrane was completed, patients entered into the follow-up period. A minimum of three follow-up visits were performed (Weeks 12 (V9), Week 18 (V10) and Week 24 (V11)). Questionnaires were administered at V9, V10 and V11: POSAS Scale Patient, POSAS Scale Physician, and Vancouver Scar Scale. The Patient's self-assessment ease of wound questionnaire was completed by the patient at Visit 11. External, surface echography of the scars of the two breasts were realised at Visits 9 and 11. See FIG. 1.

The biophotonic silicone membrane was used seven days after the surgery and was administered as per the following: i) remove the dressings, if any, and cleanse the wound/scar with normal saline irrigation; ii) apply the biophotonic silicone membrane on the post-surgical wound/scar of the breast randomly selected to be treated with the biophotonic silicone membrane (the biophotonic silicone membrane should cover all of the wound/scar following the breast reduction surgery, including the horizontal, vertical and peri-areolar wound/scar); if possible, the biophotonic silicone membrane should cover approximately 1 cm of healthy skin all around the wound/scar; if the size of the biophotonic silicone membrane is too large, it can be carefully cut to the appropriate size, using sterile scissors; the non-adherent side of the biophotonic silicone membrane was applied on the wounds at Treatment visits 1, 2, and 3; the adherent side was applied on Treatment visits 4, 5, 6, 7, and; at all Treatment Visits (Visits 1 to 8), the breast being treated with the biophotonic silicone membrane was illuminated two consecutive times for 5 minutes (automatic timer) for a total of 10 minutes with a break period (no illumination) of 1 to 2 minutes between illuminations. The multi-LED lamp as described herein was positioned such that the illumination was performed at a distance of 5 cm from the wound/scar. The same biophotonic silicone membrane was used for the two illuminations; the maximum width illuminated by the multi-LED lamp is 18cm, should the illumination not capture the entire treatment area, an additional illumination, following the same procedure described above, is authorized to treat the remaining area only. Should an additional illumination be required, the first area will be protected with a white protective cloth during the additional illumination. As part of the treatment, the biophotonic silicone membrane should remain on the wound/scar in between treatment visits after the last illumination of Visit 4 until Visit 8.

The treatment started at Day 7 post-surgery, with an authorized visit window of 7 days, meaning that the first treatment was initiated 7 to 14 days after surgery. The following treatment frequency was used for the wound/scar treated with the biophotonic silicone membrane: Visits 1 to 4 (first 2 weeks of treatments)-10 minutes' illumination twice a week: apply a new biophotonic silicone membrane on the wound/scar; illuminate 5 minutes the biophotonic silicone membrane with the multi-LED lamp; wait 1-2 minutes after the end of the first illumination period; illuminate again 5 minutes the same biophotonic silicone membrane with the multi-LED lamp. At the 4^{th} treatment, leave the biophotonic silicone membrane on the wound/scar after the 2 illumination periods, until the next treatment at Week 3/Visit 5. Visits 5 to 8 (4 last weeks of treatments)-10 minutes' illumination once a week: apply a new biophotonic silicone membrane on the wound/scar; illuminate 5 minutes the biophotonic silicone composition with the multi-LED lamp; wait 1-2 minutes after the end of the first illumination period; illuminate again 5 minutes the same biophotonic silicone membrane with the multi-LED lamp; leave the biophotonic silicone membrane on the wound/scar after the two illumination periods, until the next treatment visit (for a maximum of 28 cumulative days). At the last treatment visit (Visit 8), remove gently the biophotonic silicone membrane after the two illumination periods. The Standard of Care (e.g., wound massaging with Vitamin E Cream) will be applied on the second breast, according to the recommendations of the surgeon.

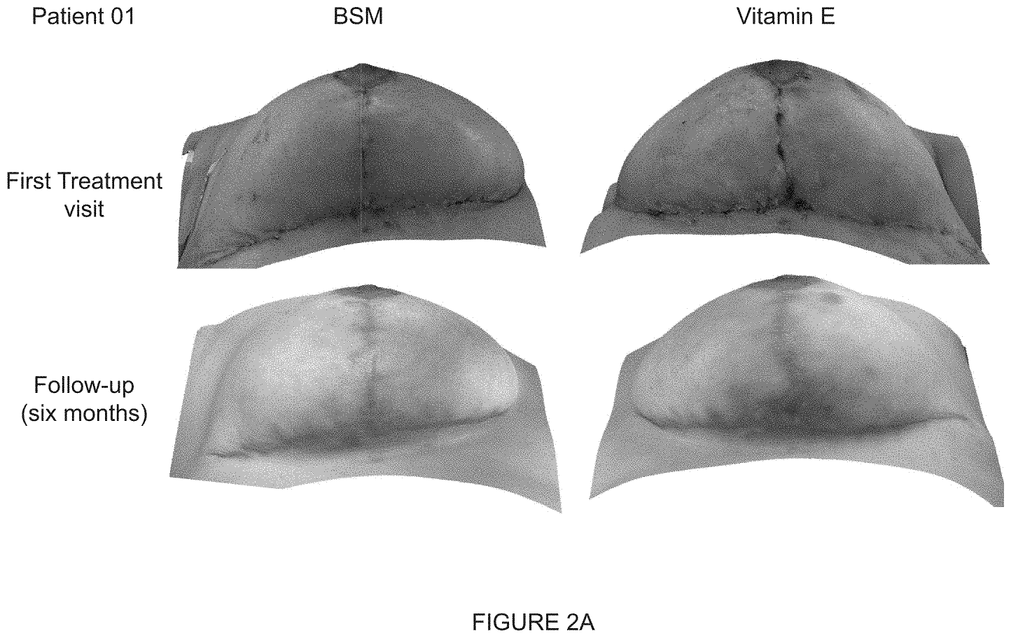

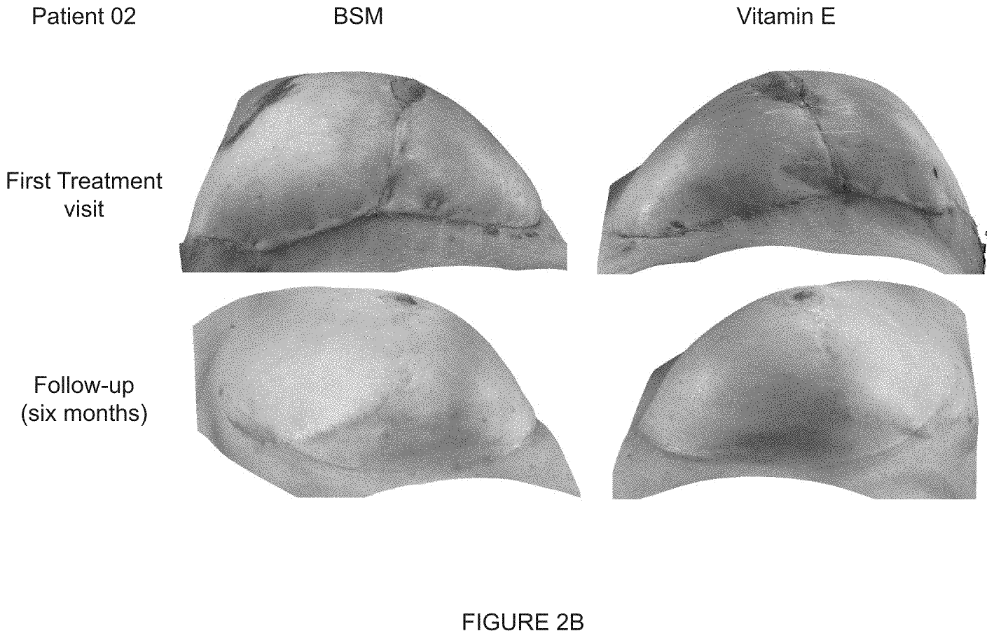

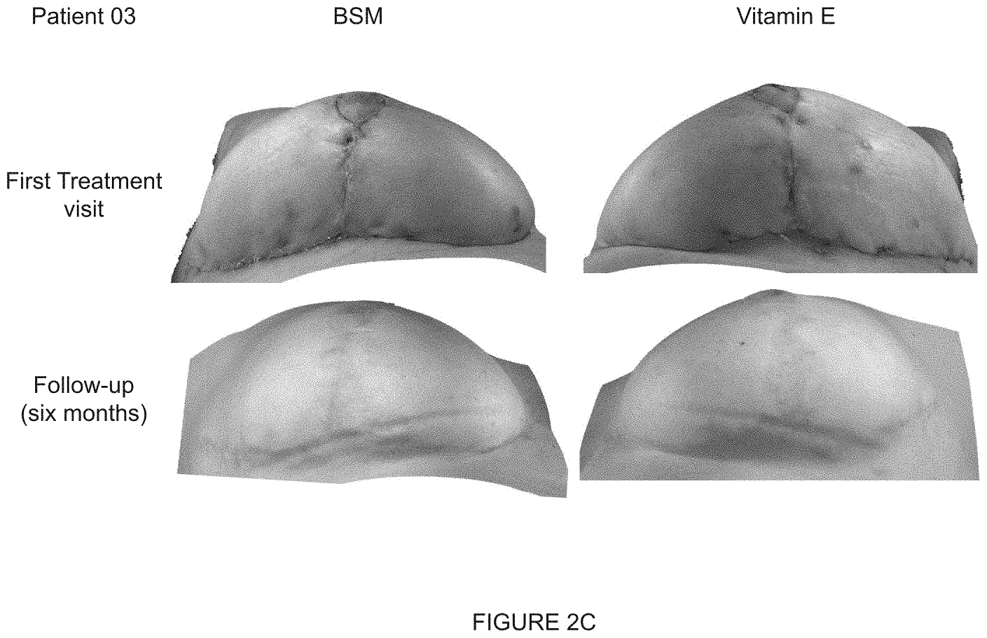

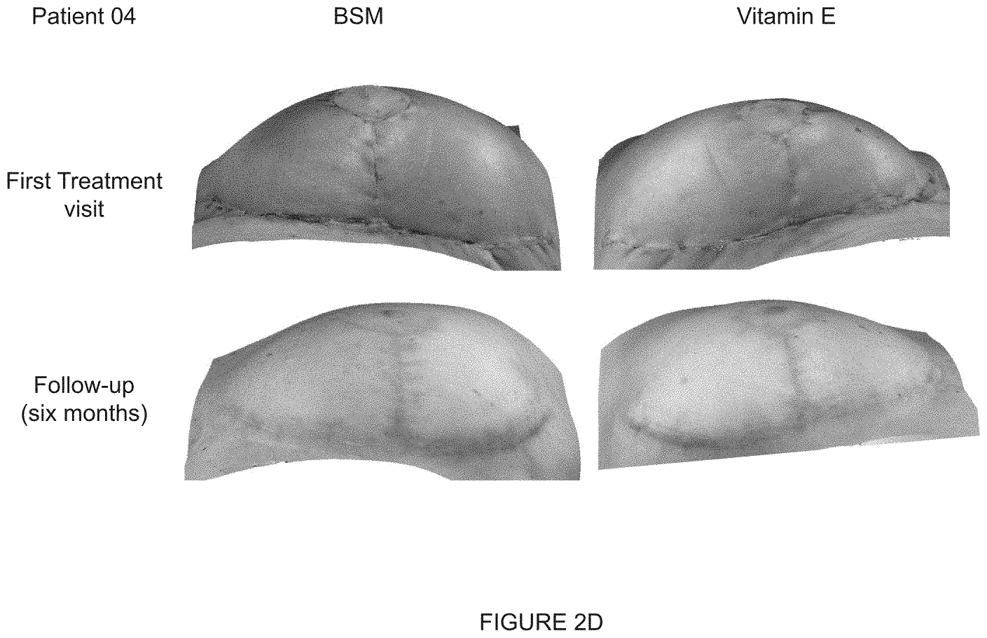

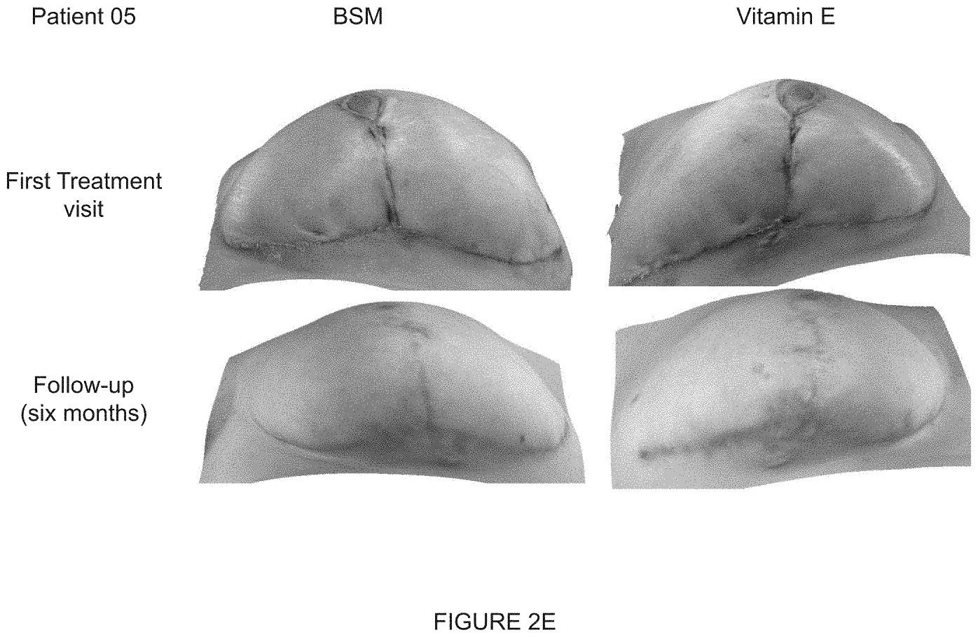

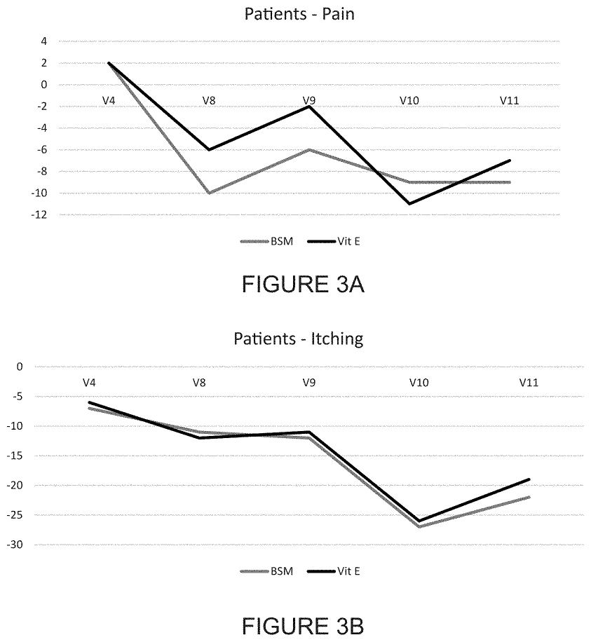

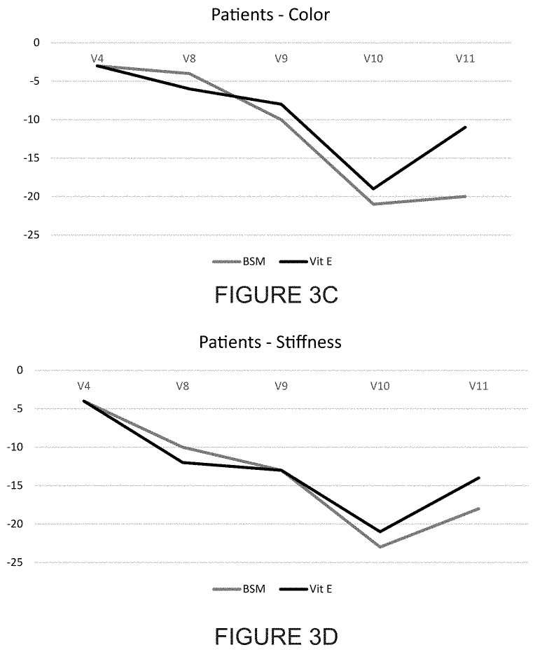

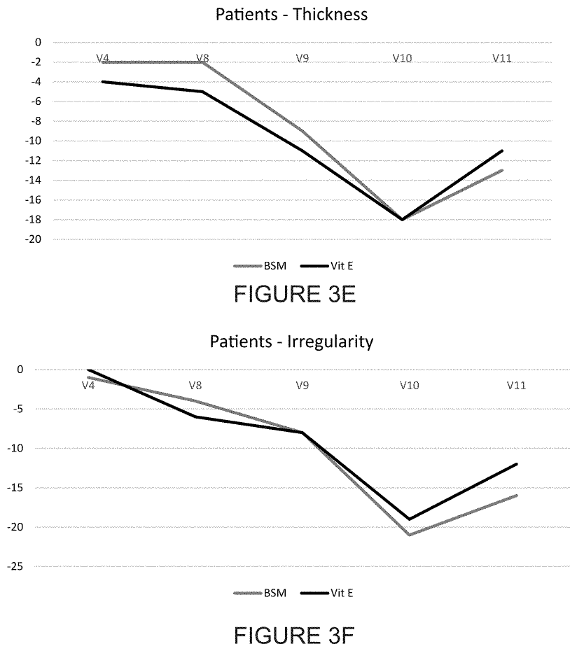

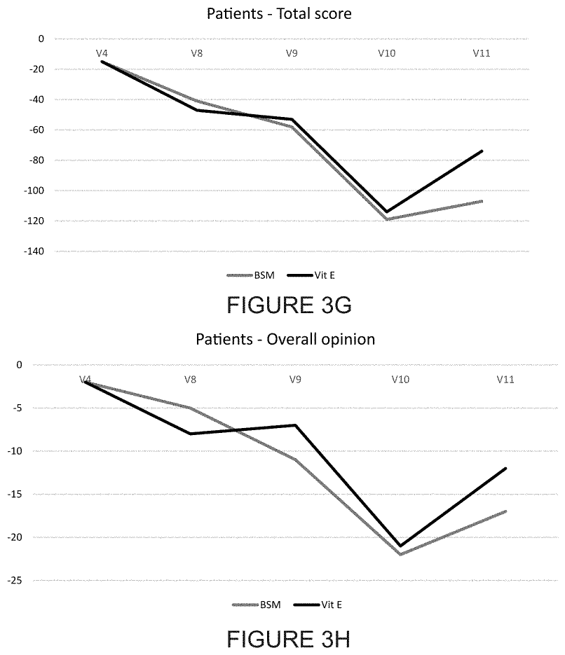

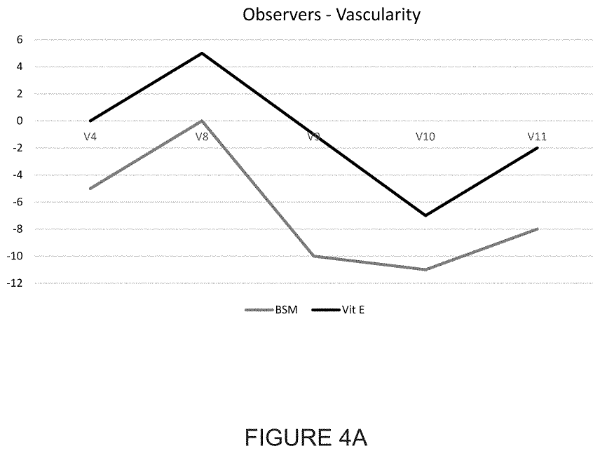

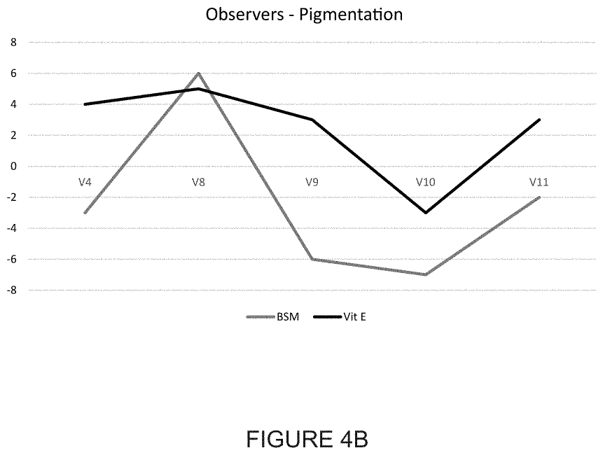

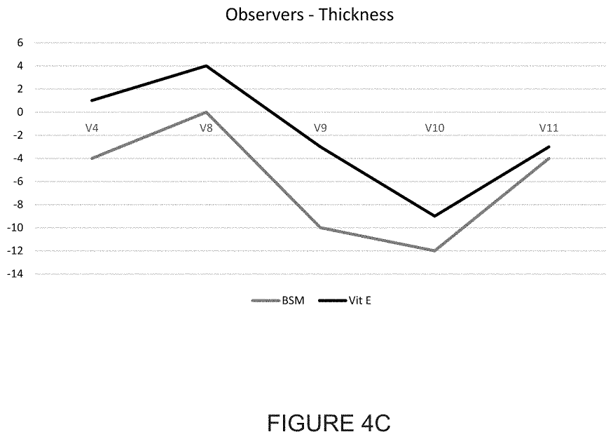

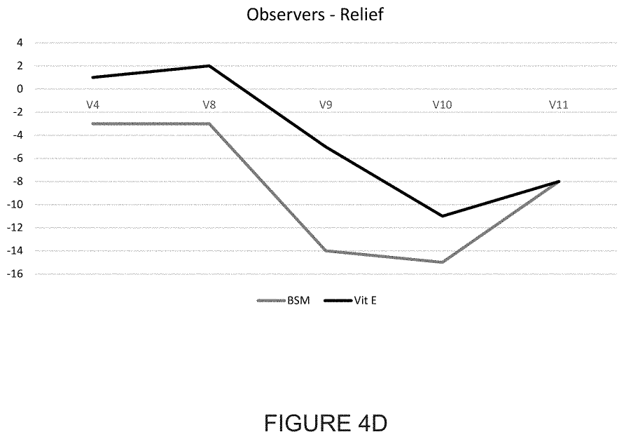

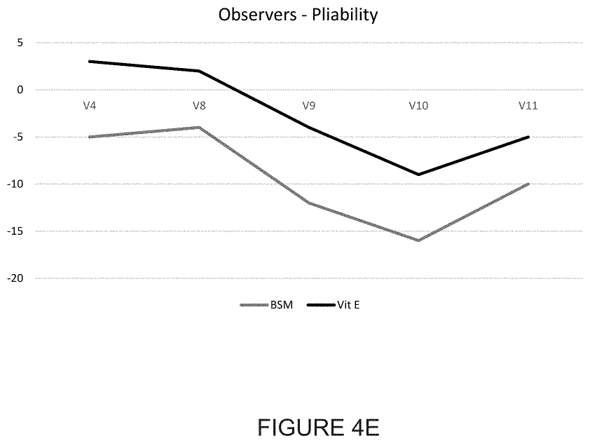

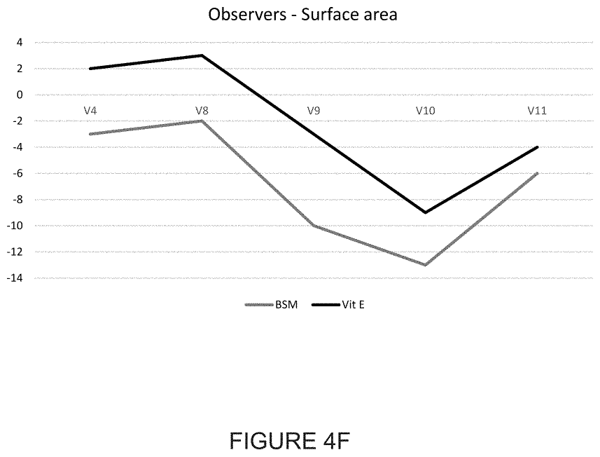

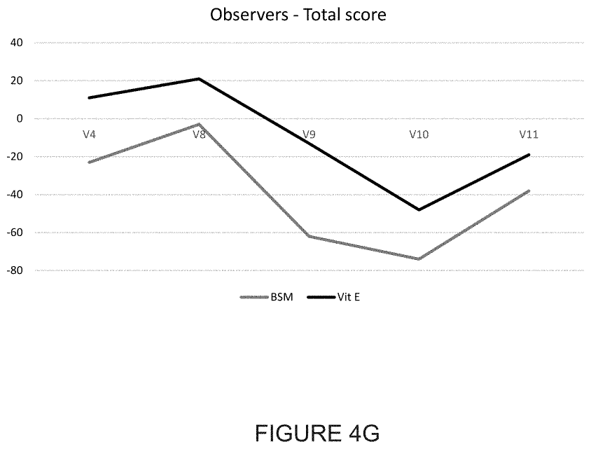

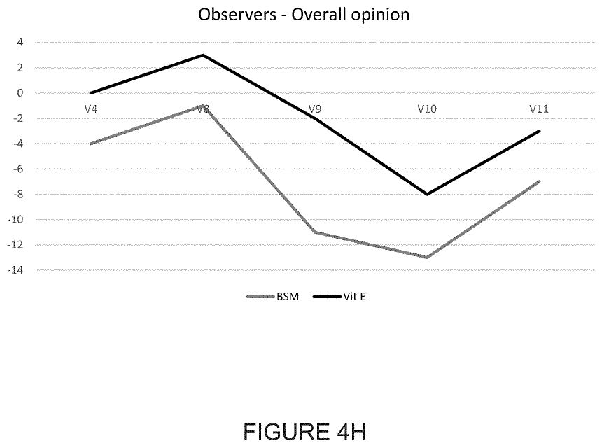

3D-photographs of the two treating areas of the wounds were taken at each of the Treatment and Follow-up visits and are presented in FIGS. 2A to 2E. The results of the study are presented in FIGS. 2A to 2E. The results show that the biophonic silicone membrane of the present technology was more efficient at treating and reducing a scar than treatment with the Standard of Care consisting on wound massages with Vitamin E cream. Similar results were obtained on the Vancouver Scar Scale (FIGS. 3A-3H) which indicated that a treatment using the biophotonic silicone membrane of the present technology was efficient at, and in some instances more efficient than the Standard of Care treatment, ameliorating at least one of: pain, itchiness, color, stiffness, and thickness of the scar area. The Patient and Observer Scar Assessment Scale (POSAS) (FIGS. 4A-4H) also demonstrates that a treatment using the biophotonic silicone membrane of the present technology was efficient at, and in some instances more efficient than the Standard of Care treatment, ameliorating at least one of: vascularity, pigmentation, thickness, relief, pliability and surface area of the scar area.

### Example 4: Biophotonic Silicone Membrane in the Treatment of Dermal Fibrosis as Well as of Other Fibroproliferative Disorders.

Under anesthesia, a full-thickness excisional wound (2.0 cm×1.5 cm) was made on the back of each mouse and a human STSG was transplanted onto the wound and secured with sutures. The surgical site was then covered with a non-adherent petrolatum (Xeroform™, Covidien, Mansfield, Mass.) and gauze in a tie over bolster dressing to apply pressure.

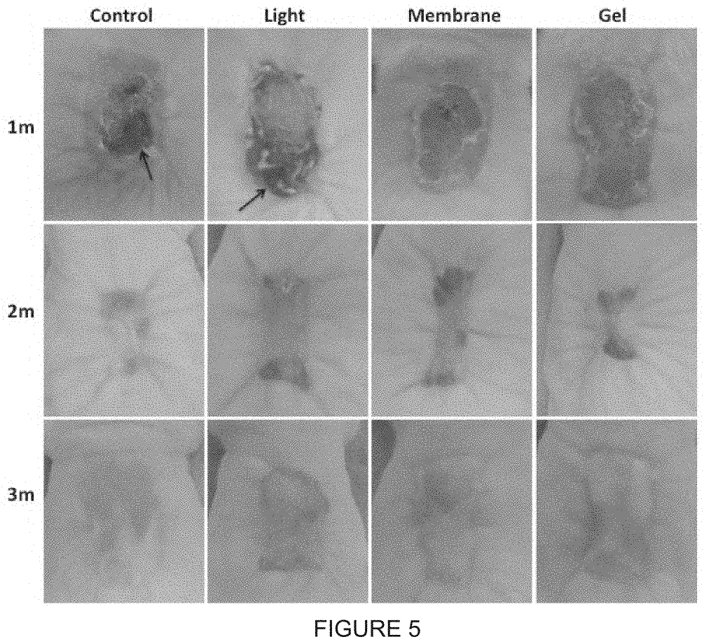

The biophotonic silicone gel consisted in solutions of Pluronic. Pluronic F-127 was dissolved in a certain volume of cold de-ionised water (˜4° C.). The concentration of Pluronic is expressed in weight per volume of H₂O. For the preparation of stock thermogelling Pluronic solution (25% w/v), a precise mass of 25.00 g of Pluronic F-127 was added, under magnetic stirring, to 100 mL of H₂O in an Erlenmeyer of 250 mL. The Erlenmeyer was then cooled in an ice bath (between 2 and 4° C.), while continuing stirring for about 1 hour, until complete dissolution of the Pluronic F-127. The resulting solution was then stored at about 4° C. The gelation test indicated that such solution turns into hydrogel after 5 min at room temperature (˜22° C.). The biophotonic silicone membrane was prepared as outlined in Example 1 above. The LED lamp used delivered a non-coherent blue light with a single peak wavelength and a maximum emission between 440-460 nm. The irradiance or power density was between 110 and 150 mW/cm² at 5 cm. The radiant fluences during a single treatment of 5 minutes was between 33 and 45 J/cm². Under anesthesia via nasal halothane, grafted wounds were treated topically with the biophotonic silicone gel (2-mm thickness), or 1.5×2 cm² of the biophotonic silicone membrane in combination with the LED lamp placed at 5 cm distance for 5 minutes, or LED lamp alone. Mice were treated twice per week during 6 consecutive weeks. Control mice (untreated mice) did not receive any treatment after grafting. Human STSGs were transplanted onto full-thickness excisional wounds on the back of mice. The wounds were then treated with the biophotonic silicone gel or membrane in combination with LED lamp twice a week for 5 min each time during 6 consecutive weeks. As controls, the wounds were treated with light alone or left untreated. They were then monitored weekly after grafting by digital photography. The morphology of wounds showed that the treatment with the biophotonic silicone membrane combination with light accelerated the wound closure 1 month after treatment (FIG. 5). Treatment with the biophotonic silicone membrane significantly reduced wound size compared to the other 3 groups at 1 month post-treatment as shown in Table 1.

TABLE 1
Wound area, % of original wounds (mean ± SEM)

| | Months Post-treatment | | | |
|---|---|---|---|---|
| | 0 | 1 | 2 | 3 |
| Negative | 100.00 ± 5.56 | 62.41 ± 8.19 | 53.69 ± 11.52 | 50.58 ± 10.40 |
| Control | n = 16 | n = 5 | n = 5 | n = 6 |
| Light | 100.00 ± 1.18 | 58.60 ± 10.87 | 55.37 ± 15.99 | 57.63 ± 10.31 |
| | n = 18 | n = 6 | n = 6 | n = 6 |
| Biophotonic | 100.00 ± 5.90 | 49.88 ± 10.70 | 45.92 ± 14.07 | 55.10 ± 10.20 |
| membrane | n = 18 | n = 6 | n = 6 | n = 6 |
| Biophotonic | 100.00 ± 6.19 | 62.34 ± 8.99 | 55.99 ± 12.12 | 56.55 ± 10.43 |
| gel | n = 17 | n = 5 | n = 6 | n = 6 |

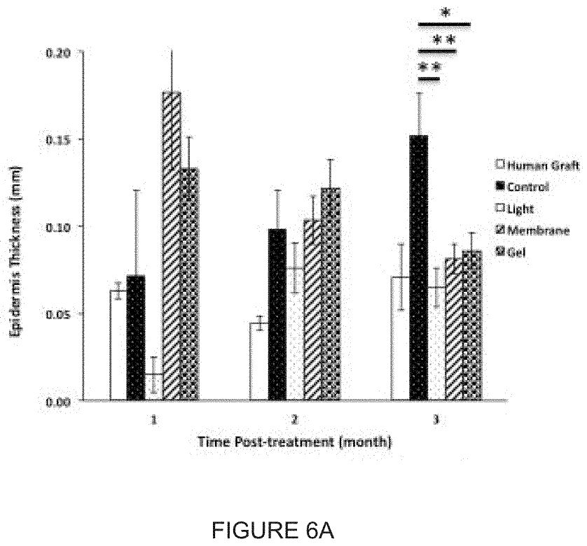

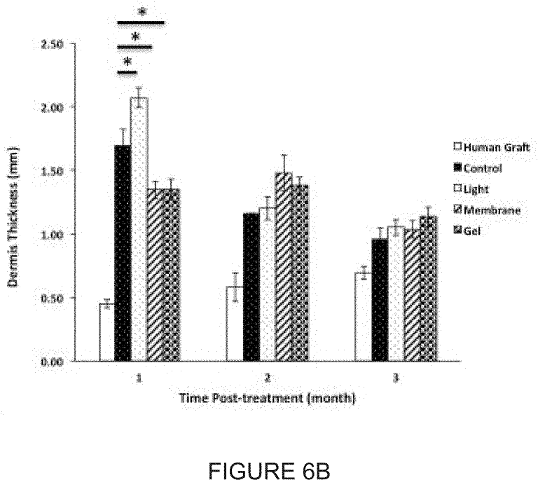

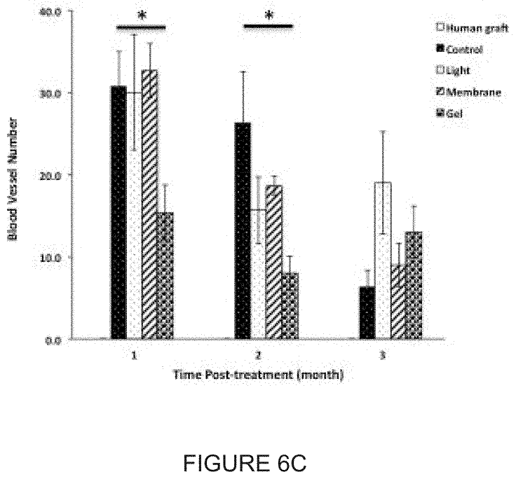

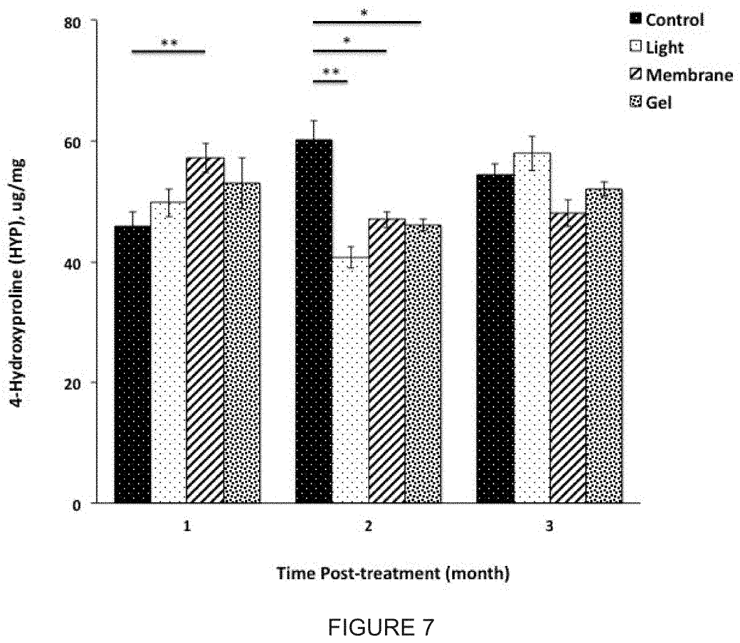

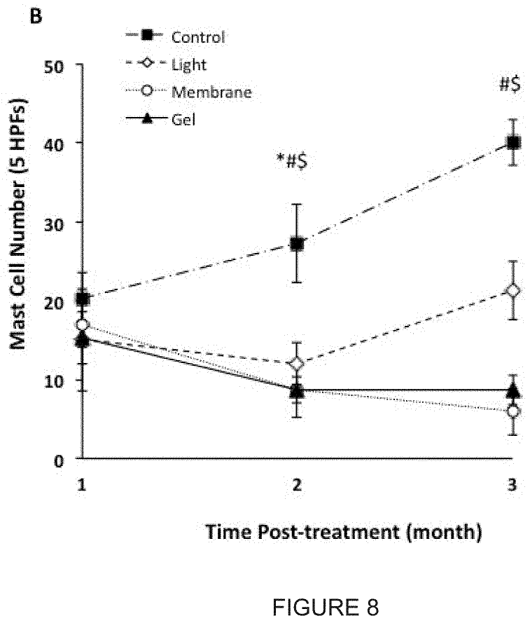

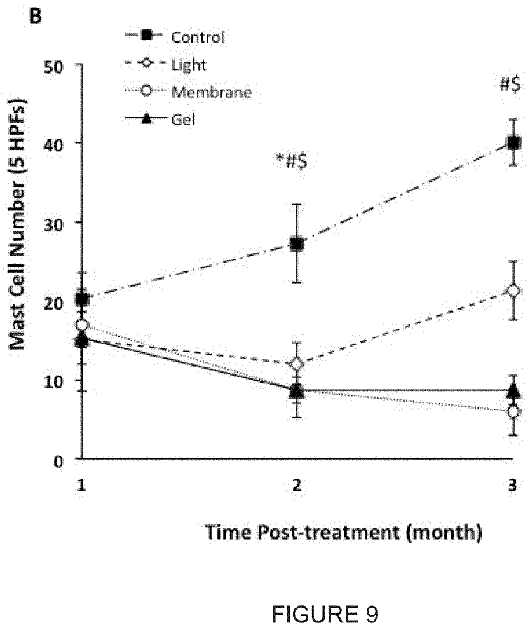

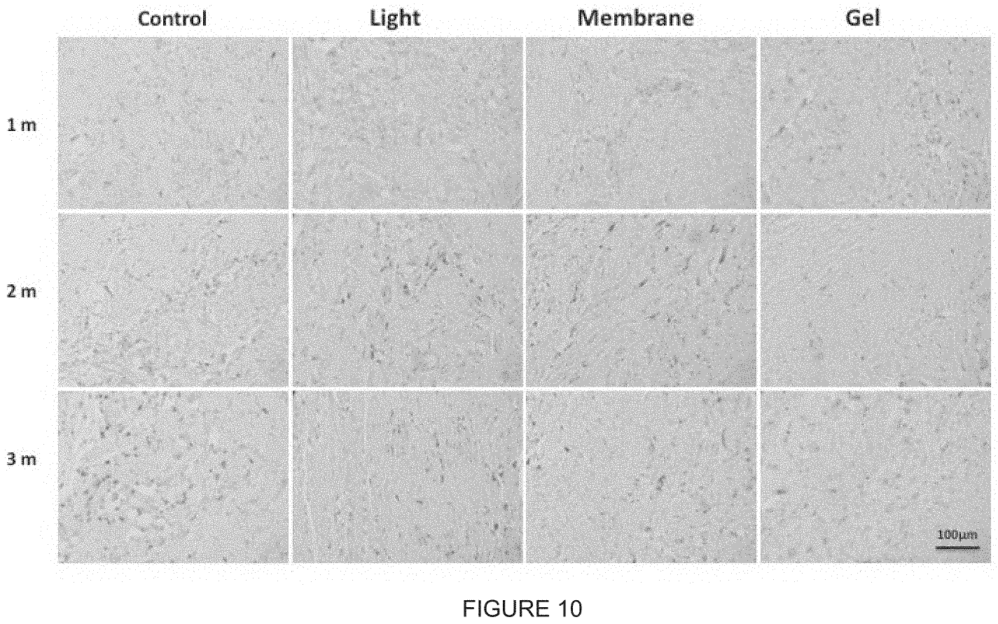

Furthermore, the scabs were almost completely gone and smooth epithelium covered the entire wounds. Light microscopy of paraffin sections of mice xenografts stained with H&E revealed that the biophotonic silicone membrane in combination with LED light treatment induced a complete reepithelization with a thicker and flatter epidermis layer 1 month after treatment as compared to the groups of light alone or untreated mice where reepithelization was delayed (2 months after treatment). Compared to the groups of untreated or light only treated mice, treatment with the biophotonic silicone membrane did significantly reduce epidermal thickness at 3 months but not 1 or 2 months post-treatment (FIGS. 6A-6C). The numbers of blood vessels were also evaluated. Blood vessel formation reduction was noticed when the mice were treated with biophotonic silicone membrane in combination with LED light during the first 2 months post-treatment, (FIGS. 6A-6C). Altogether, these data showed that treatment with the biophotonic silicone membrane did enhance reepithelization together with reducing scar thickness and formation of blood vessels. Collagen was also quantified in the mice xenografts using a 4-hydroxyproline assay. The data demonstrated that collagen deposition was significantly reduced in the treated mice with the biophotonic silicone membrane plus light, compared to untreated mice at 2 months post-treatment (FIG. 7). The role of the biophotonic silicone mebrane on myofibroblast accumulation was examined by quantifying these cells in the dermis of xenografts using αSMA staining. Our result demonstrated that the treatment with biophotonic silicone membrane in combination with LED light significantly decreased the number of myofibroblasts in xenograft tissues at 1 and 3 months post-treatment while light alone treatment promoted myofibroblast accumulation in the first 2 months after treatment as compared to non-treatment (FIG. 8). Furthermore, recent studies have highlighted the importance of mast cells in mediator release, cell proliferation, and collagen remodelling during wound healing, with high numbers of activated mast cells being associated with scarring. FIG. 9 showed that a significant reduction of toluidine blue stained mast cells in the xenografts of mice treated with wound membrane or wound gel plus light was observed at 2 and 3 months post-treatment. Nonetheless, the light alone treatment downregulated mast cells accumulation only 2 months after treatment. Finally, the CTGF, an important fibrotic growth factor in scar formation, was also quantified as described above. FIG. 10 showed that although CTGF expression was not modulated among all the groups at 1 month post-treatment, CTGF expression was significantly reduced in the wound gel plus light treated group at 2 and 3 months after treatment.

Altogether, these findings provide the evidence that the biophotonic silicone membrane of the present technology has the potential of prevention and/or treatment of dermal fibrosis as well as of other fibroproliferative disorders.

It should be appreciated that the technology is not limited to the particular embodiments described and illustrated herein but includes all modifications and variations falling within the scope of the technology as defined in the appended claims.

1. A biophotonic silicone membrane for use in management of a scar in a subject, the biophotonic silicone membrane comprising: a silicone phase and a surfactant phase, wherein the surfactant phase comprises at least one light-absorbing molecule solubilized in a surfactant.

2. The biophotonic silicone membrane of claim 1, further comprising an adherent side and a non-adherent side.

3. The biophotonic silicone membrane of claim 1 or 2, wherein the scar is post-surgical scar.

4. The biophotonic silicone membrane of any one of claims 1 to 3, wherein the light-absorbing molecule is a xanthene dye.

5. The biophotonic silicone membrane of claim 4, wherein the xanthene dye is selected from Eosin Y, Eosin B, Erythrosine B, Fluorescein, Rose Bengal and Phloxin B.

6. The biophotonic silicone membrane of any one of claims 1 to 5, wherein the light has a peak wavelength between about 400 nm and about 750 nm.

7. The biophotonic silicone membrane of any one of claims 1 to 6, wherein the surfactant phase is emulsified in the silicone phase.

8. The biophotonic silicone membrane method of any one of claims 1 to 7, wherein the surfactant comprises a block copolymer.

9. The biophotonic silicone membrane of claim 8 wherein the block copolymer comprises at least one hydrophobic block and at least one hydrophilic block.

10. The biophotonic silicone membrane of claim 9, wherein the surfactant phase comprises a surfactant which is thermogellable.

11. The biophotonic silicone membrane of any one of claims 1 to 10, wherein the surfactant is water soluble.

12. The biophotonic silicone membrane of any one of claims 1 to 11 wherein the surfactant comprises at least one sequence of polyethylene glycol-propylene glycol ((PEG)-(PPG)).

13. The biophotonic silicone membrane of any one of claims 1 to 12, wherein the surfactant is a poloxamer.

14. The biophotonic silicone membrane of any one of claims 1 to 13, wherein the silicone phase comprises a soft adhesive silicone.

15. The biophotonic silicone membrane of claim 14, wherein the content of the soft adhesive silicone in the silicone phase is 5-100 wt %.

16. The biophotonic silicone membrane of claim 14 or 15, wherein the silicone phase further comprises a low consistency silicone or a clear low consistency silicone.

17. The biophotonic silicone membrane of any one of claims 1 to 16, comprising about 60-95 wt % silicone phase and about 5-40 wt % surfactant phase, or about 80 wt % silicone phase and about 20 wt % surfactant phase.

18. The biophotonic silicone membrane of any one of claims 1 to 17, wherein the surfactant comprises at least one sequence of (PEG)-(PLA) or (PEG)-(PLGA) or (PEG)-(PCL).

19. The biophotonic silicone membrane of any one of claims 1 to 18, wherein the biophotonic silicone membrane is coated with a layer of soft adhesive silicone.

20. The biophotonic silicone membrane of any one of claims 1 to 18, wherein the silicone in the silicone phase comprises an organopolysiloxane having silicone-bonded alkenyl groups.

21. The biophotonic silicone membrane of claim 20, wherein the organopolysiloxane having silicone-bonded alkenyl groups is dimethylsiloxane capped at both molecular termini with vinyldimethylsilyl groups.

22. The biophotonic silicone membrane of any one of claims 1 to 21, wherein the silicone in the silicone phase comprises an organohydrogensiloxane having an average of two or more silicone-bonded hydrogen atoms in the molecule.

23. The biophotonic silicone membrane of claim 22, wherein the organohydrogensiloxane having an average of two or more silicone-bonded hydrogen atoms in the molecule is dimethylsiloxane and methyl hydrogen siloxane capped at both molecular termini with trimethylsilyl groups.

24. The biophotonic silicone membrane of any one of claims 1 to 23, the silicone in the silicone phase is a silicone elastomer having one or more of: (i) a Shore-A hardness of from about 20 to about 45 as measured in accordance with ASTM D2240 using a type A durometer hardness tester; (ii) a breaking elongation of at least about 800% as measured in accordance with ASTM D412; and (iii) a tensile strength of at least about 15.0 MPa.

25. A method for preventing or treating a scar in a subject in need thereof comprising:
a) placing a biophotonic silicone membrane over a target skin tissue, wherein the biophotonic silicone membrane comprises a silicone phase and a surfactant phase, and wherein the surfactant phase comprises at least one light-absorbing molecule solubilized in a surfactant; and
b) illuminating said biophotonic silicone membrane with light having a wavelength that overlaps with an absorption spectrum of the at least one light-absorbing molecule.

26. The method of claim 25, wherein steps a) and b) are performed at least once weekly.

27. The method of claim 25, wherein steps a) and b) are performed at least twice weekly.

28. The method of any one of claims 25 to 27, wherein the light in step b) is illuminated for 5 minutes at two consecutive intervals.

29. The method of claim 28, wherein the two consecutive intervals are separated by a period comprising 1 to 2 minutes without illumination.

30. The method of claim 25, wherein the light in step b) is illuminated for 5 minutes followed by a period of 1 minute without illumination followed by a further illumination period of 5 minutes.

31. The method of any one of claims 25 to 30, wherein the biophotonic silicone membrane comprises an adherent side and a non-adherent side.

32. The method of any one of claims 25 to 31, wherein the target skin tissue is post-surgical skin tissue.

33. The method of any one of claims 25 to 32, wherein the scar is any one or more of a hypertrophic scar, a keloid, a linear scar, a sunken scar, or a stretched scar.

34. The method of any one of claims 25 to 33, wherein the biophotonic silicone membrane is removed after illumination.

35. The method of any one of claims 25 to 34, wherein the biophotonic silicone membrane is left in place after illumination.

36. The method of any one of claims 25 to 35, wherein the light-absorbing molecule at least partially photobleaches after illumination.

37. The method of any one of claims 25 to 35, wherein the light-absorbing molecule photobleaches after illumination.

38. The method of any one of claims 25 to 37, wherein the composition is illuminated until the light-absorbing molecule is at least partially photobleached.

39. The method of any one of claims 25 to 38 wherein the light-absorbing molecule can absorb and/or emit light in the visible range.

40. The method of any one of claims 25 to 39, wherein the light-absorbing molecule is a xanthene dye.

41. The method of claim 40, wherein the xanthene dye is selected from Eosin Y, Eosin B, Erythrosine B, Fluorescein, Rose Bengal and Phloxin B.

42. The method of any one of claims 25 to 41, wherein the light has a peak wavelength between about 400 nm and about 750 nm.

43. The method of any one of claims 25 to 42, wherein the light has a peak wavelength between about 400 nm and about 500 nm.

44. The method of any one of claims 25 to 43, wherein the surfactant phase is emulsified in the silicone phase.

45. The method of any one of claims 25 to 44, wherein the surfactant comprises a block copolymer.

46. The method of claim 45, wherein the block copolymer comprises at least one hydrophobic block and at least one hydrophilic block.

47. The method of claim 46, wherein the surfactant phase comprises a surfactant which is thermogellable.

48. The method of any one of claims 25 to 47, wherein the surfactant is water soluble.

49. The method of any one of claims 25 to 48, wherein the surfactant comprises at least one sequence of polyethylene glycol-propylene glycol ((PEG)-(PPG)).

50. The method of any one of claims 25 to 49, wherein the surfactant is a poloxamer.

51. The method of any one of claims 25 to 50, wherein the silicone phase comprises a soft adhesive silicone.

52. The method of claim 51, wherein the content of the soft adhesive silicone in the silicone phase is 5-100 wt %.

53. The method of claim 51 or 52, wherein the silicone phase further comprises a low consistency silicone or a clear low consistency silicone.

54. The method of any one of claims 25 to 53, comprising about 60-95 wt % silicone phase and about 5-40 wt % surfactant phase, or about 80 wt % silicone phase and about 20 wt % surfactant phase.

55. The method of any one of claims 25 to 54, wherein the surfactant comprises at least one sequence of (PEG)-(PLA) or (PEG)-(PLGA) or (PEG)-(PCL).

56. The method of any one of claims 25 to 55, wherein the biophotonic silicone membrane is coated with a layer of soft adhesive silicone.

57. A kit comprising a biophotonic silicone membrane having a silicone phase and a surfactant phase, and wherein the surfactant phase comprises at least one light-absorbing molecule solubilized in a surfactant; and instructions for performing the method of any one of claims 25 to 56.

58. The kit of claim 57, further comprising a multi-LED lamp.

59. A biophotonic silicone membrane for use in preventing and/or treating a scar in a subject, the biophotonic silicone membrane comprising: a silicone phase and a surfactant phase, wherein the surfactant phase comprises at least one light-absorbing molecule solubilized in a surfactant.
